(19) United States
(12) Patent Application Publication
(10) Pub. No.: US 2025/0000979 A1
(43) Pub. Date: Jan. 2, 2025

(54) TRIPLET-TRIPLET ENERGY TRANSFER WITH LIGHT EXCITATION AT LONG WAVELENGTHS AND METHODS THEREOF

(71) Applicant: The University of Hong Kong, Hong Kong (CN)

(72) Inventors: Weiping Wang, Hong Kong (CN); Kaiqi Long, Hong Kong (CN)

(73) Assignee: The University of Hong Kong, Hong Kong (CN)

(21) Appl. No.: 18/737,027

(22) Filed: Jun. 7, 2024

Related U.S. Application Data
(60) Provisional application No. 63/506,991, filed on Jun. 8, 2023.

(51) Int. Cl. A61K 41/00; A61K 31/69

(52) U.S. Cl. CPC A61K 41/0071; A61K 31/69

(57) ABSTRACT

The present disclosure generally relates to various phototriggered drug release and photoreactions, including reactions generally based on triplet-triplet energy transfer with light excitation at long wavelengths. One aspect of the present disclosure is directed to systems and methods for absorbing energy in a photosensitizer, transferring that energy by triplet-triplet energy transfer to cleave a cleavable or other active moiety causing the release of a releasable moiety. One aspect relates to a composition comprising a photosensitizer which is capable of singlet-to-triplet (S-T) activation at a near-infrared (NIR) wavelength, a cleavable moiety to accept triplet-triplet energy transfer from the photosensitizer in a higher energy state to cause cleavage of the cleavable moiety, and a releasable moiety releasable from the composition upon cleavage of the cleavable moiety. Such systems and methods may be used in various biological or physical applications. Also disclosed are methods for making or using such systems, kits including such systems.

### CROSS-REFERENCE TO RELATED APPLICATION

This application claims priority to U.S. Provisional Application No. U.S. 63/506,991, filed on Jun. 8, 2023, the contents of which are incorporated herein by reference in their entirety.

### 1. FIELD

The present disclosure generally relates to various phototriggered drug release and photoreactions, including reactions generally based on triplet-triplet energy transfer with light excitation.

### 2. BACKGROUND

Photolysis, also called as photo-uncaging or photocleavage reaction, has been utilized for controlling molecular functions or release of desired components with light irradiation.¹ It has been identified as a powerful approach with spatiotemporally controllable manner and showed excellent strengths in imaging, photocatalysis, photopharmacology, neuroscience, and drug delivery. Photocleavable prodrugs (PD), with tailor-made structures composed of photoremovable protecting groups (PPGs) and drug molecules, have been developed for light-triggered precise drug activation.^{2, 3} After systematic administration of photocleavable prodrugs, local light irradiation can be applied onto disease lesions to specifically activate the prodrugs in situ, reducing systemic toxicity and thus increasing biocompatibility and therapeutic efficacy.^{4, 5} In biomedical applications, near-infrared (NIR) light (650-900 nm) is highly desirable for photoactivated therapy, due to its deep tissue penetration and low phototoxicity.^{6, 7} However, the low photon energy of NIR light usually cannot meet the direct activation threshold of the commonly used PPGs, such as PPGs based on nitrobenzene⁸, coumarin⁹⁻¹¹ and boron-dipyrromethene (BODIPY)¹².

One of the strategies to achieve long-wavelength light-triggered prodrug photolysis is to develop photon upconversion systems. Lanthanide-doped upconversion nanoparticles (UCNPs) have emerged as reliable platforms for turning NIR light into UV/visible light, thus enabling long-wavelength light to activate short-wavelength light-responsive PPGs.^{13, 14} However, the required excitation power density is usually high (10¹-10⁴ W/cm²), since UCNPs exhibit low absorption coefficient and cross-sections, and the efficiency of luminescence resonance energy transfer (LRET) between UCNP and PPGs remains unsatisfactory. Triplet-triplet annihilation-based upconversion (TTA-UC) is another strategy for long-wavelength light-triggered photolysis, which depends on multi-step energy transfer between photosensitizer and annihilator to produce upconverted photons (FIG. 1A).^{15, 16} TTA-UC enabled the utilization of low-irradiance long-wavelength light (10⁻³-10⁻¹ W/cm²), however, the internal energy consumption during the multi-step energy transfer processes still resulted in low quantum yields and photolysis efficiency in many cases.

To overcome these concerns, a “one-photon upconversion-like photolysis” strategy can sensitize the prodrug to its triplet excited state with a single low-energy photon (FIG. 1B).^{17, 18} As the mechanism, long-wavelength light activates photosensitizer (PS) from ground state to singlet excited state (S), followed by intersystem crossing (ISC) to its triplet excited state (T₁) and the activation of prodrug through triplet-triplet energy transfer. This process is like the photon upconversion, but only single photon is involved. So far, the anti-Stocks shift of this process is still limited (that is, we only can use red light to activate green light-responsive photolysis), since the photon energy needs to be higher than the S₁ state of PS, and only the PS with low singlet state energy level can be used (e.g., platinum- or palladium-coordinated porphyrins). Thus, it is important to develop a new mechanism of upconversion-like photolysis that overcame the limitation of photon energy which must be higher than the S₁ state of PS by utilizing PS with singlet-to-triplet (S-T) absorption (STPS).

### 3. SUMMARY

The present disclosure generally relates to various phototriggered drug release and photoreactions based on triplet-triplet energy transfer with light excitation, preferably direct singlet to triplet activation of a photosensitizer at near-infrared (NIR) wavelengths.

In one aspect, the present invention is generally directed to a composition. In one set of embodiments, the composition comprises a photosensitizer which is capable of singlet-to-triplet (S-T) activation at a near-infrared (NIR) wavelength, a cleavable moiety capable of accepting triplet-triplet energy transfer from the photosensitizer in a higher energy state to cause cleavage of the cleavable moiety, and a releasable moiety releasable from the composition upon cleavage of the cleavable moiety.

In some embodiments, the present disclosure provides a composition comprising a photosensitizer which is capable of singlet-to-triplet (S-T) activation at a near-infrared (NIR) wavelength, and a prodrug comprising a cleavable moiety and a releasable moiety, wherein the cleavable moiety accepts triplet-triplet energy transfer from the photosensitizer in a higher energy state to cause cleavage of the cleavable moiety and release of the releasable moiety.

In some embodiments, the composition comprises a carrier material comprising a photosensitizer, and a prodrug comprising an active moiety (e.g., a drug), and a releasable moiety. In some embodiments, absorption of an incident photon by the photosensitizer causes energy transfer to the photosensitizer and then to the active moiety to cause a chemical reaction within the active moiety. In some embodiments, the active moiety is a prodrug comprising a cleavable moiety and a releasable moiety. In one set of embodiments, the composition comprises a photosensitizer able to absorb a photon at near infrared (NIR) wavelength, leading to singlet-to-triplet (S-T) activation to produce a higher energy state, followed by a triplet-triplet energy transfer from the photosensitizer to the cleavable moiety in the active moiety, so as to cause cleavage of the cleavable moiety, and release of releasable moiety from the composition. According to another set of embodiments, the composition comprises a photosensitizer able to directly sensitize a cleavable moiety via triplet-triplet transfer process (TTET).

In one embodiment, the composition comprises an annihilator. In one embodiment, the composition does not comprise an annihilator.

The present invention, in another aspect, is generally drawn to a method. In accordance with one set of embodiments, the method includes absorbing a photon by a photosensitizer at near infrared-NIR wavelength to cause singlet-to-triplet (S-T) activation of the photosensitizer, transferring energy from the photosensitizer directly to an active moiety via triplet-triplet energy transfer, producing an excited state of a cleavable moiety in the active moiety via triplet-triplet energy transfer, and causing a chemical reaction in the active moiety using the transferred energy. In some embodiments, the photosensitizer is activated from a singlet (S₀) state to a triplet (T₁) state. In some embodiments, the photosensitizer is activated directly from a singlet (S₀) state to a triplet (T₁) state.

The method, in another set of embodiments, includes applying, to a subject, a composition comprising a photosensitizer, a cleavable moiety able to accept triplet-triplet energy transfer from the photosensitizer to cause cleavage of the cleavable moiety, and applying light to at least a portion of the subject to cause cleavage of the cleavable moiety.

The method, in another set of embodiments, includes applying, to a subject, a composition comprising a photosensitizer capable of singlet-to-triplet (S-T) activation upon exposure to a near-infrared (NIR) wavelength; and a prodrug comprising a cleavable moiety and a releasable moiety, wherein the cleavable moiety accepts triplet-triplet energy transfer from the photosensitizer in a higher energy state to cause cleavage of the cleavable moiety and release of the releasable moiety; and applying near-infrared (NIR) light to the subject to cause the cleavage of the cleavable moiety and release of the releasable moiety. In some embodiments, the triplet-triplet energy is not transferred via an annihilator.

In still another set of embodiments, the method includes applying, to a subject, a composition comprising a photosensitizer, a cleavable moiety, and a carrier material, and applying light at a near-infrared (NIR) wavelength to the subject. In some embodiments, absorption of light by the photosensitizer from a singlet to triplet (S-T) state causes energy transfer from the triplet state of the photosensitizer to the cleavable moiety to cause cleavage of the cleavable moiety and release of the releasable moiety.

In still another set of embodiments, the method includes applying, to a tumor in a subject, a composition comprising a photosensitizer, a prodrug comprising a cleavable moiety and a releasable moiety, and a carrier material, and applying light to at least a portion of the tumor, wherein absorption of light by the photosensitizer causes energy transfer to the cleavable moiety to cause cleavage of the cleavable moiety and release of the releasable moiety. In one embodiment, the releasable moiety is a drug. In one embodiment, the releasable moiety comprises a drug. In one embodiment, the releasable moiety is an anti-cancer drug.

In some embodiments applicable to all compositions and methods of the present disclosure, the photosensitizer is activated from a singlet (S₀) state to a triplet (T₁) state. In some embodiments, the photosensitizer is activated directly from a singlet (S₀) state to a triplet (T₁) state. In some embodiments, the triplet-triplet energy transfer is from the triplet (T₁) state of the photosensitizer to the cleavable moiety. In some embodiments, the triplet (T₁) state of the photosensitizer is higher than the triplet state of the cleavable moiety.

In some embodiments applicable to all compositions and methods of the present disclosure, the photosensitizer is activated from a singlet to triplet (S-T) state at a near infrared (NIR) wavelength or upon exposure to a near infrared (NIR) light, which is preferably from 650-900 nm. In some embodiments, the near infrared (NIR) light is at a wavelength of between about 650 nm and about 750 nm. In some embodiments, the near infrared (NIR) light is at a wavelength of about 690 nm. In some embodiments, the photosensitizer has an excitation wavelength of between about 650 nm and about 750 nm. In some embodiments, the photosensitizer has an excitation wavelength of about 690 nm.

In some embodiments applicable to all compositions and methods of the present disclosure, the photosensitizer comprises a transition metal-porphyrin. In some embodiments, the transition metal is osmium (Os). In one embodiment, the photosensitizer is Os (II) bromophenyl terpyridine complex (Os(bptpy)₂²⁺).

In some embodiments, the cleavable moiety in the prodrug is photocleavable. In some embodiments, the cleavable moiety is BODIPY. In some embodiments, the prodrug comprises a drug that is linked to BODIPY.

In some embodiments, the releasable moiety in the prodrug is a drug. In some embodiments, the releasable moiety in the prodrug comprises a drug or a derivative of a drug. In some embodiments, the drug is an anti-inflammatory drug, an anti-cancer drug or an anti-angiogenesis drug. In some embodiments, the drug is selected from the group consisting of chlorambucil (CAB), vadimezan, indomethacin, naproxen, ibuprofen, benzyloxycinnamic acid, tetracaine, dopamine, tyramine and homoveratrylamine.

In another aspect, the present invention encompasses methods of making one or more of the embodiments described herein, for example, compositions comprising photosensitizers and a cleavable moiety. In still another aspect, the present invention encompasses methods of using one or more of the embodiments described herein, for example, compositions comprising photosensitizers and a cleavable moiety.

### 4. BRIEF DESCRIPTION OF THE DRAWINGS

The patent or application file contains at least one drawing executed in color. Copies of this patent or patent application publication with color drawing(s) will be provided by the Office upon request and payment of the necessary fee.

FIGS. 1A-D. Schematic illustration of the mechanisms of the reported photolysis strategies and the NIR light-triggered upconversion-like photolysis with one-step energy transfer in this work. (A) The mechanism of TTA-UC-mediated photolysis. (B) The mechanism of red light-triggered upconversion-like photolysis. (C) The mechanism of NIR light-triggered upconversion-like photolysis with one-step energy transfer. (D) S1 and T1 energy levels and chemical structures of Os(bptpy)₂²⁺ and BODIPY-Cb. (PS: photosensitizer; An: annihilator; PD: prodrug).

FIGS. 2A-F. (A) Normalized absorption spectra of BODIPY-Cb and Os(bptpy)₂²⁺ in dichloromethane. (B) Phosphorescence quenching of Os(bptpy)₂²⁺ (10 μM) in the presence of different concentrations of BODIPY-Cb, ex=492 nm, in N₂-saturated toluene. (C) Stern-Volmer plot based on the phosphorescence quenching by different concentrations of BODIPY-Cb. (D) Photolysis reaction of BODIPY-Cb with/without Os(bptpy)₂²⁺ (denoted as Os). (E) Photolysis rate of BODIPY-Cb upon 690 nm light irradiation for different time durations. (F) Generation rate of Cb upon 690 nm light irradiation for different time durations. (Solution: N₂-saturated methanol; BODIPY-Cb concentration: 10⁻³ M; Light irradiance: 690 nm, 100 mW/cm²; n=3).

FIGS. 3A-B. Upconversion-like photolysis of different prodrugs in the presence of Os(bptpy)₂²⁺ and 690 nm light irradiation. (A) The reaction and conditions of the photolysis reaction. (B) Chemical structures of the BODIPY prodrugs. The percentage presents the photolytic yield of respective prodrug.

FIGS. 4A-F. (A) Schematic illustration of NIR light-triggered drug release from the nanoparticle (Os/BC NP). (B) Size distribution of various nanoparticles. (C) TEM image of Os/BC NPs. (D) Normalized absorption of BC NPs and Os/BC NPs in aqueous solutions. The red area indicates the NIR window. (E) Percentage of uncleaved BODIPY-Cb and released Cb from Os/BC NPs upon 690 nm light irradiation for 0-30 min (n=3). (F) Percentage of uncleaved BODIPY-Cb and released Cb from BC NPs upon 690 nm light irradiation for 0-30 min (n=3).

FIGS. 5A-E. (A) Schematic illustration of Os NP, BC NP and Os/BC NP, and their Jablonski diagrams of energy transfer processes upon NIR-light irradiation. (B) Cytotoxicity of Os NPs, BC NPs and Os/BC NPs in the dark against HeLa cells. (C) Cytotoxicity of Os NPs, BC NPs and Os/BC NPs with light irradiation against HeLa cells (n=5). (D) Calcein-AM/PI staining of HeLa cells after treatment with Os NPs, BC NPs and Os/BC NPs with light irradiation. Scale bar: 20 μm. (E) Apoptosis study of HeLa cells treated with Os NPs, BC NPs and Os/BC NPs with light irradiation. Light irradiation: 690 nm, 100 mW/cm², 30 min.

FIGS. 6A-H. (A) Representative IVIS fluorescence images of the mice after injection of free DiR and DiR NPs within 24 h (n=3). Red dashed circles indicate tumor areas. (B) Quantitative analysis of biodistribution in major organs and tumor determined by IVIS. Tu, He, Lu, Sp, Li, and Ki represent tumor, heart, lung, spleen, liver, and kidney, respectively. ***p<0.005. (C) Schematic illustration of the treatment schedule. (D) Photograph of a mouse irradiated with NIR light at the tumor site. (E) Tumor volume of each group (n=5) (Lamp parameter: 690 nm, 300 mW/cm², 10 min). (F) Photograph of tumors resected at Day 13 after different treatments. (G) Body weight of each group. (H) Representative H&E staining of tumor sections of different treatment groups. Scale bar: 200 μm.

FIGS. 7A-B. (A) Photolysis rate of BODIPY-Cb upon 530 nm light irradiation for different time durations in air- or N₂-saturated solutions. (B) Generation rate of Cb upon 530 nm light irradiation for different time durations in air- or N₂-saturated solution. (BODIPY-Cb concentration: 10⁻³ M; Light irradiance: 50 mW/cm²; n=3).

FIG. 8. HPLC elution curve of BODIPY-Cb with/without NIR light irradiation in the exist of 0.1 of Os(bptpy)₂²⁺. (Prodrug concentration: 10⁻³ M; lamp parameter: 690 nm, 100 mW/cm², 30 min; time duration: 0-8 min; solution: 88% methanol, 10% dichloromethane, 2% acetone, N₂-saturated; λ_{abs}=260 nm).

FIG. 9. HPLC elution curve of free DMXAA, BODIPY-DMXAA with/without NIR light irradiation in the exist of 0.1 eq Os(bptpy)₂²⁺. (Prodrug concentration: 10⁻³ M; lamp parameter: 690 nm, 100 mW/cm², 30 min; time duration: 5 min; solution: 88% methanol, 10% dichloromethane, 2% acetone, N₂-saturated; λ_{abs}=254 nm).

FIG. 10. HPLC elution curve of free IDM, BODIPY-IDM with/without NIR light irradiation in the exist of 0.1 eq Os(bptpy)₂²⁺. (Prodrug concentration: 10⁻³ M; lamp parameter: 690 nm, 100 mW/cm², 30 min; time duration: 5 min; solution: 88% methanol, 10% dichloromethane, 2% acetone, N₂-saturated, λ_{abs}=254 nm).

FIG. 11. HPLC elution curve of free NPX, BODIPY-NPX with/without NIR light irradiation in the exist of 0.1 eq Os(bptpy)₂²⁺. (Prodrug concentration: 10⁻³ M; lamp parameter: 690 nm, 100 mW/cm², 30 min; time duration: 5 min; solution: 88% methanol, 10% dichloromethane, 2% acetone, N₂-saturated; λ_{abs}=260 nm).

FIG. 12. HPLC elution curve of free IBF, BODIPY-IBF with/without NIR light irradiation in the exist of 0.1 eq Os(bptpy)₂²⁺. (Prodrug concentration: 10⁻³ M; lamp parameter: 690 nm, 100 mW/cm², 30 min; time duration: 5 min; solution: 88% methanol, 10% dichloromethane, 2% acetone, N₂-saturated; λ_{abs}=254 nm).

FIG. 13. HPLC elution curve of free BCA, BODIPY-BCA with/without NIR light irradiation in the exist of 0.1 eq Os(bptpy)₂²⁺. (Prodrug concentration: 10⁻³ M; lamp parameter: 690 nm, 100 mW/cm², 30 min; time duration: 5 min; solution: 88% methanol, 10% dichloromethane, 2% acetone, N₂-saturated; λ_{abs}=254 nm).

FIG. 14. HPLC elution curve of free TCI, BODIPY-TCI with/without NIR light irradiation in the exist of 0.1 eq Os(bptpy)₂²⁺. (Prodrug concentration: 10⁻³ M; lamp parameter: 690 nm, 100 mW/cm², 30 min; time duration: 5 min; solution: 88% methanol, 10% dichloromethane, 2% acetone, N₂-saturated; λ_{abs}=280 nm).

FIG. 15. HPLC elution curve of free DPA, BODIPY-DPA with/without NIR light irradiation in the exist of 0.1 eq Os(bptpy)₂²⁺. (Prodrug concentration: 10⁻³ M; lamp parameter: 690 nm, 100 mW/cm², 30 min; time duration: 5 min; solution: 88% methanol, 10% dichloromethane, 2% acetone, N₂-saturated; λ_{abs}=280 nm).

FIG. 16. HPLC elution curve of free TyrA, BODIPY-TyrA with/without NIR light irradiation in the exist of 0.1 eq Os(bptpy)₂²⁺. (Prodrug concentration: 10⁻³ M; lamp parameter: 690 nm, 100 mW/cm², 30 min; time duration: 5 min; solution: 88% methanol, 10% dichloromethane, 2% acetone, N₂-saturated; λ_{abs}=280 nm).

FIG. 17. HPLC elution curve of free HVA, BODIPY-HVA with/without NIR light irradiation in the exist of 0.1 eq Os(bptpy)₂²⁺. (Prodrug concentration: 10⁻³ M; lamp parameter: 690 nm, 100 mW/cm², 30 min; time duration: 5 min; solution: 88% methanol, 10% dichloromethane, 2% acetone, N₂-saturated; λ_{abs}=280 nm).

FIG. 18. HPLC elution curve of free Cb, BODIPY2-Cb with/without NIR light irradiation in the exist of 0.1 eq Os(bptpy)₂²⁺. (Prodrug concentration: 10⁻³ M; lamp parameter: 690 nm, 100 mW/cm², 30 min; time duration: 5 min; solution: 88% methanol, 10% dichloromethane, 2% acetone, N₂-saturated; λ_{abs}=260 nm).

FIG. 19. Size and PDI of Os/BC NPs in PBS for 72 h under 37° C.

FIG. 20. Representative fluorescent images of the tumor and major organs 24 h after the injections of free DiR and DiR-labelled NPs.

FIG. 21. Tumor weights at the end of the in vivo test (Day 13).

FIG. 22. H&E staining of major organs sections of the mice after different treatments. Scare bar: 200 μm.

FIG. 23. ¹H-NMR spectrum of Os(bptpy)₂·2PF₆.

FIG. 24. MALDI-TOF MS spectrum of Os(bptpy)₂·2PF₆.

FIG. 25. ¹H-NMR spectrum of BODIPY-Cb.

FIG. 26. ¹H-NMR spectrum of BODIPY-DMXAA.

FIG. 27. ¹H-NMR spectrum of BODIPY-IDM.

FIG. 28. ¹H-NMR spectrum of BODIPY-NPX.

FIG. 29. ¹H-NMR spectrum of BODIPY-IBF.

FIG. 30. ¹H-NMR spectrum of BODIPY-BCA.

FIG. 31. ¹H-NMR spectrum of BODIPY-TCI.

FIG. 32. ¹H-NMR spectrum of BODIPY-DPA.

FIG. 33. ¹H-NMR spectrum of BODIPY-TyrA.

FIG. 34. ¹H-NMR spectrum of BODIPY-HVA.

FIG. 35. ¹H-NMR spectrum of BODIPY2-Cb.

FIGS. 36A-F. (A-E) HPLC trace of BODIPY-OH photocage under 530 nm green light, 625 nm red light (with/without PtTPBP) and 690 nm NIR light (with/without Os(bptpy)₂²⁺) (100 mW/cm², 0-7 min) (DAD detector, 540 nm). (F) Normalized remaining amount of BODIPY-OH in different groups.

### 5. DETAILED DESCRIPTION

Prodrug photolysis enables spatiotemporal control of drug release at the desired lesions. For photoactivated therapy, near-infrared (NIR) light is preferable due to its deep tissue penetration and low phototoxicity. However, most of the photocleavable groups cannot be directly activated by NIR light. Provided herein is a new upconversion-like process via only one step of energy transfer for NIR light-triggered prodrug photolysis. A photosensitizer can be activated via singlet-triplet (S-T) absorption and achieve photolysis of boron-dipyrromethene (BODIPY)-based prodrugs via triplet-triplet energy transfer. Using the strategy, NIR light can achieve green light-responsive photolysis with a single-photon process. A wide range of drugs and bioactive molecules were designed and demonstrated to be released under low-irradiance NIR light (100 mW/cm2, 5 min) with high yields (up to 87%). Moreover, a micellar nanosystem encapsulating both photosensitizer and prodrug was developed to demonstrate the practicality of the disclosure in normoxia aqueous environment for cancer therapy. This disclosure is useful to advance the development of photocleavable prodrugs and photoresponsive drug delivery systems for photoactivated therapy.

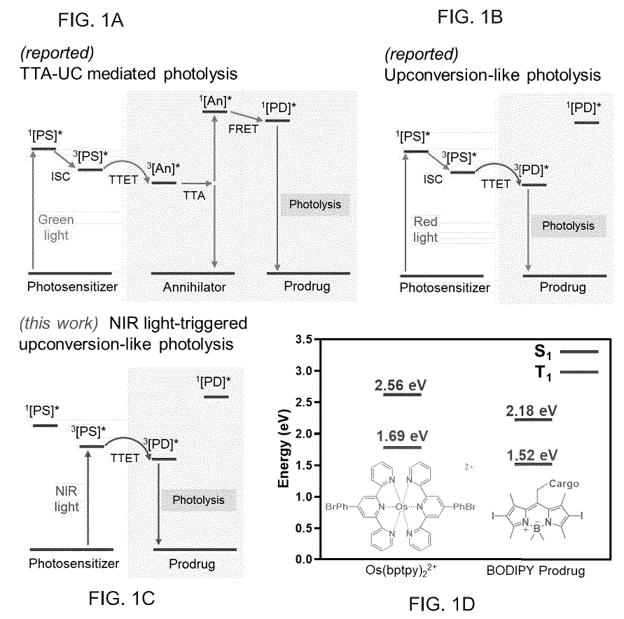

Disclosed herein is a new mechanism of upconversion-like photolysis that overcame the limitation of photon energy which must be higher than the S1 state of PS by utilizing PS with singlet-to-triplet (S-T) absorption (STPS). Since STPS can be directly activated to the T₁ state from S₀ state, the photon with energy higher than T₁ of STPS is enough to initiate the upconversion-like photolysis, greatly reducing the excitation energy level to achieve NIR light activation (FIG. 1C). Multiple steps of energy transfer can be bypassed, resulting in less internal energy loss and higher photolysis efficiency. In the presence of STPS, a series of short-wavelength light-responsive photocage-conjugated prodrugs, including the prodrugs of chlorambucil, vadimezan, indomethacin, naproxen, ibuprofen, benzyloxycinnamic acid, tetracaine, dopamine, tyramine and homoveratrylamine, was uncaged by NIR light with low irradiance and short duration (100 mW/cm2, 5 min) at high yields (up to 87%). Surprisingly, the yields of drug release were even higher than that directly triggered by short-wavelength light. The strategy was verified in vivo by using a micellar nanosystem for light-triggered drug release and photoactivable cancer therapy. This study provides a new strategy to utilize the low-energy photons of long-wavelength light to trigger prodrug photolysis with high yield, which demonstrates potentials for advanced photoactivated therapy.

The present disclosure generally relates to various photoreactions, including reactions generally based on triplet-triplet energy transfer process (TTET) from photosensitive and photocleavable groups. One aspect of the present disclosure is directed to systems and methods for absorbing energy (e.g., from a photon) in a photosensitizer from a singlet to a triplet state (preferably from a S₀ to a T₁ state), transferring that energy by triplet-triplet energy transfer to a prodrug comprising a cleavable moiety and a releasable moiety, to result in cleavable of the cleavable moiety and release of the releasable moiety. In some embodiments, the releasable moiety is or comprises a drug.

In some embodiments, the photosensitizer and the cleavable moiety may be contained within a suitable carrier material, for example, a particle or a micelle. Such systems and methods can be used in a variety of applications, including various biological or physical applications. For example, such systems and methods can be useful for delivering drugs or other releasable moieties to regions of the body in a subject. Other aspects of the present invention are generally directed to methods for making or using such systems, kits including such systems, or the like.

It should be understood that, as is known to those of ordinary skill in the art, the term “triplet” generally refers to the electronic state of a molecule, not to the number of electrons that are present within the molecule. For example, in a triplet state, the molecule may have unpaired electrons present such that the net spin the molecule has is 1. Absorption of energy by a molecule, e.g., through absorption of a photon, may result in an electron from the molecule being “raised” from a lower energy state (or shell) to a higher energy state (or shell), which may alter the net spin of the molecule, while emission or transfer of that energy may allow a higher-energy electron to return to a lower state. In some embodiments, the absorption of a photon causes an electron from the molecule to be raised from a singlet (S) stage to a triplet (T) stage. In some embodiments, the singlet stage is a ground stage, for example S₀ stage. In some embodiments, the triplet state is a T₁ state. In some embodiments, the photosensitizer is activated directly from a S₀ state to a T₁ stage.

In some embodiments, the energy from the triplet state of the photosensitizer may be transferred to a cleavable moiety in a prodrug that comprises a cleavable moiety and a releasable moiety. A variety of mechanisms may be involved in the transfer of such energy, such as triplet-triplet energy transfer (TTET). For instance, triplet-triplet energy transfer may be accomplished through the exchange of electrons that carry different spin and energy, e.g., between two molecules (such as between the cleavable moiety and a photosensitizer). The cleavable moiety may then be cleaved as a result of the energy from the photosensitizer, and preferably releases the releasable moiety which is preferably a drug. The energy transfer to the cleavable moiety may occur through a variety of processes.

In some embodiments, a prodrug contains a cleavable moiety and a releasable moiety. In some embodiments, cleavage of the cleavable moiety can cause breakage of one or more bonds (e.g., covalent bonds) within or linked to the cleavable moiety. In some embodiments, cleavage of the cleavable moiety causes breakage of a bond between the cleavable moiety and releasable moiety. In some embodiments, cleavage of the cleavable moiety may cause a portion of the moiety to become separated from e.g., as releasable moiety. Thus, in such a fashion, absorption of a photon (e.g., via a photosensitizer) may produce a chain of events that results in the release of a releasable moiety, for example a drug. Accordingly, by controlling the incident light, the release of releasable moiety can be controlled as desired. However, it should be understood that a releasable moiety is not required, for example, cleavage of the cleavable moiety may result in other chemical or structural changes within the cleavable moiety. In addition, it should be understood that the energy may be transferred to other active moieties instead of a cleavable moiety, e.g., the energy may result in photoisomerization, rearrangement, photocycloaddition, or other chemical reactions.

Thus, in one set of embodiments, a composition comprising a photosensitizer and a cleavable moiety (or other active moiety) may be applied to a region (e.g., within a sample, within a subject, etc.), and near infrared (NIR) light applied to the region (or at least a portion of the region) in order to cause cleavage of the cleavable moiety, for example, to cause a chemical change, to release a releasable moiety, or the like. As mentioned, other active moieties may also be used. For example, if the active moiety is a cleavable moiety, the releasable moiety may be a drug, and light may be applied to thereby cause release of the drug. In one embodiment, the transferred energy causes photoreaction of the active moiety (e.g., photoisomerization, rearrangement, photocleavage, or other chemical reactions). In one embodiment, the photoreaction further triggers the structural or compositional change of the carrier material containing the composition.

In certain embodiments, the encapsulated drugs or other molecules/objects can be released. As another non-limiting example, the releasable moiety can be a tracer (for example, a radioactive tracer, an inert molecule, a detectable entity, etc.) that can be introduced to a system (e.g., a biological system such as a cell or an organism, or a non-biological system such as a polymer), and the tracer released at an appropriate time (e.g., through applying light), for instance, instead of being instantly released upon administration or incorporation of the composition. The tracer may then be detected using any suitable technique, e.g., fluorescence, radioactivity, biological assay, chemical or enzymatic activity, etc.

In some embodiments, components such as the photosensitizer and/or the cleavable moiety may be contained within a suitable carrier material using physical encapsulation or chemical conjugation. In some embodiments, the components are on the surface of the carrier material. In some embodiments, the components are part of the carrier material. In some embodiments, the carrier material may hold the photosensitizer and/or the cleavable moiety in close proximity to each other, e.g., to allow for electron and/or photon transfers to occur as discussed herein. For example, in one embodiment, the photosensitizer and/or the cleavable moiety may be contained within a particle, such as a microparticle or a nanoparticle. In some embodiments, the particle may contain an environment (e.g., a hydrophobic or nonpolar environment), for instance, to keep the photosensitizer or the cleavable moiety in close proximity, to facilitate transfer of electrons and/or photons, etc.

### 5.1.1 Photosensitizer

In some embodiments of the present disclosure, the composition includes a photosensitizer. The photosensitizer can be any composition that is able to absorb a photon to produce a higher energy state. The energy may be transferrable to the cleavable moiety. In some embodiments, the photosensitizer is capable of absorbing at wavelength of near-infrared (NIR) light, e.g., from about 650 nm to about 900 nm. In some embodiments, the photosensitizer has an excitation wavelength at near infrared (NIR) energy, e.g., from about 650 nm to about 900 nm. As non-limiting examples, the photosensitizer may have an excitation wavelength (i.e., the photosensitizer is activated or undergoes singlet to triplet (S-T) activation) at a near infrared (NIR) wavelength of about 650 nm-700 nm, about 700-750 nm, about 750 nm-800 nm, about 800 nm-850 nm, or about 850 nm-900 nm.

In some embodiments, the photosensitizer is capable of absorbing at near-infrared (NIR) light (i.e., has an excitation wavelength at near infrared (NIR) energy) e.g., at about 600-650 nm, about 610-620 nm, about 620-630 nm, about 630-640 nm, about 640-650 nm, about 650-660 nm, about 660-670 nm, about 670-680 nm, about 680-690 nm, about 690-700 nm, about 700-710 nm, about 710-720 nm, about 720-730 nm, about 730-740 nm, about 740-750 nm, about 750-760 nm, about 760-770 nm, about 770-780 nm, about 780-790 nm, about 790-800 nm, about 800-810 nm, about 810-820 nm, about 820-830 nm, about 830-840 nm, about 840-850 nm, about 850-860 nm, about 860-870 nm, about 870-880 nm, about 880-890 nm, about 890-900 nm, or about 900 nm.

It should be understood that the photosensitizer can be excited by light of a single wavelength (e.g., monochromatic light, such as would be supplied by a laser), or by light of different wavelengths (e.g., from a light source producing a spectrum of wavelengths).

In some embodiments, the photosensitizer is a fluorophore. In some embodiments, the photosensitizer comprises a transition metal. In some embodiments, the photosensitizer is a transition metal capable of singlet-to-triplet (S-T) activation at near infrared (NIR) wavelength. In some embodiments, the photosensitizer is a transition metal capable of singlet-to-triplet (S₀-T₁) activation at near infrared (NIR) wavelength. In some embodiments, the photosensitizer is a transition metal capable of direct singlet-to-triplet (S₀-T₁) activation at near infrared (NIR) wavelength, i.e., an electron is transferred from a ground state (S₀) directly to a triplet (e.g., T₁) state. In some embodiments, the near infrared (NIR) wavelength is 690 nm.

In some embodiments, a transition metal capable of near infrared (NIR) absorption or activation is Osmium (Os) or its complexes. In some embodiments, a transition metal capable of near infrared (NIR) absorption or activation is titanium (Ti), zirconium (Zr), vanadium (V) or nickel (Ni) or their complexes.

In one embodiment, the photosensitizer comprises a porphyrin or a porphyrin derivative, e.g., a transition metal-porphyrin such as an Os porphyrin. Specific non-limiting examples of photosensitizers include Osmium porphyrins such as Os (II) bromophenyl terpyridine complex (Os(bptpy)₂²⁺), Bis(2,2′;6′,2″-terpyridine)osmium (II) hexafluorophosphate, Bis(4′-(pyridin-4-yl)-2,2′:6′,2″-terpyridine)osmium (II) hexafluorophosphate, Bis(4,4′,4″-tri-tert-butyl-2,2′:6′,2″-terpyridine)osmium (II) hexafluorophosphate, Bis(4′-phenyl-2,2′:6′,2″-terpyridine)osmium (II) hexafluorophosphate, or Bis(4′-(4-methoxyphenyl)-2,2′:6′,2″-terpyridine) osmium (II) hexafluorophosphate.

A variety of triplet photosensitizers are known to those of ordinary skill in the art; many of these are commercially available.

In some embodiments, the yield of the releasable moiety obtained by photolysis at near infrared (NIR) wavelengths is higher than the yield obtained when a photosensitizer absorbing at green-light or red-light irradiation or red lights are used. For example, in some embodiments, activation of the photosensitizer at near infrared (NIR) wavelength (i.e., by exposure to light at a near infrared (NIR) wavelength, e.g., 690 nm) leads to release of about 50%-55% of the cleavable moiety (e.g., a drug). In some embodiments, about 55%-60%, 60%-65%, 65%-70%, 70%-75%, 75%-80%, 80%-81%, 81%-82%, 82%-83%, 83%-84%, 84%-85%, 85%-86%, 86%-87%, 87%-88%, 88%-89%, 89%-90%, 90%-91%, 91%-92%, 92%-93%, 93%-94%, 94%-95%, 95%-96%, 96%-97%, 97%-98%, 98%-99%, 99%, or about 100% of the cleavable moiety is released from the composition upon irradiation of the photosensitizer at a near infrared (NIR) wavelength.

For example, as contemplated herein, near infrared (NIR) light radiation of BODIPY-Cb using Os(bptpy)₂²⁺ as a photosensitizer led to 84.17±4.21% release of free chlorambucil (Cb). In contrast, photolysis by green-light irradiation (530 nm) or red light activation (625 nm) led to much lower yields of about 31.71% and 41.74%, respectively.

### 5.1.2 Cleavable and Releasable Moieties

In some embodiments, the cleavable moiety accepts triple-triplet energy transfer from the photosensitizer. In some embodiments, the photosensitizer in the triplet state is in a higher energy state than the cleavable moiety in the triplet state. In some embodiments, the cleavable moiety accepts triplet-triplet energy transfer to cause cleavage of the cleavable moiety and release of the releasable moiety.

In some embodiments, the cleavable moiety is photocleavable. In some embodiments, the cleavable moiety comprises a boron-dipyrromethene (BODIPY) or a BODIPY derivative, or fullerene or a fullerene derivative. In some embodiments, the cleavable moiety is a BODIPY derivative represented by the structure:

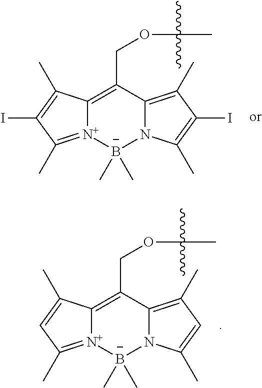 [SMILES: *OCC1=C2C(C)=C(I)C(C)=[N+]2[B-](C)(C)n2c(C)c(I)c(C)c21 ; "or" ; *OCC1=C2C(C)=CC(C)=[N+]2[B-](C)(C)n2c(C)cc(C)c21]

In some embodiments, the transfer of energy to the active moiety results in the cleavage of a bond within or linked to the cleavable moiety, and/or within or linked to a different portion of the cleavable moiety, for example the releasable moiety.

In some embodiments, the composition comprises a prodrug, the prodrug comprising the cleavable moiety and the releasable moiety. In some embodiments, the composition comprises a therapeutically-effective amount of the prodrug.

In some embodiments, provided herein is a prodrug comprising a cleavable moiety and a releasable moiety.

In some embodiments, a prodrug provided according to some embodiments of the present disclosure comprises a BODIPY derivative. In some embodiments, a prodrug provided according to some embodiments of the present disclosure is represented by the structure:

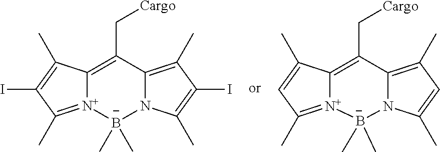

wherein “Cargo” represents the releasable moiety. In some embodiments, “Cargo” comprises a drug. In some embodiments, Cargo is derived from a drug.

In some embodiments, the cleavable moiety (e.g., BODIPY or a BODIPY derivative) is bonded to the drug via a cleavable chemical group such as an ester, a carbamate, a carbonate, an amide or the like. In some embodiments, the prodrug is a BODIPY-ester prodrug represented by the structure:

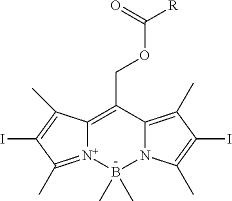

wherein R is a drug or wherein R is derived from a drug.

In some embodiments, the prodrug is a BODIPY-carbamate prodrug represented by the structure:

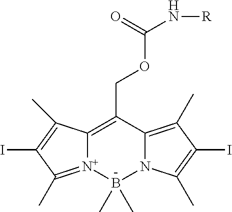

wherein R is a drug or wherein R is derived from a drug.

In some embodiments, the releasable moiety and the cleavable moiety are linked through a chemical group such as an ester, a carbamate, a carbonate, an amide and the like. Cleavable of a bond in the chemical group (e.g., ester, carbamate), can cause the release of the releasable moiety which can be a therapeutic agent or a drug. However, it should be understood that in other embodiments, the cleavage of a single bond does not necessarily require the release of a releasable moiety, for instance, if more than one bond connects portions of the molecule together. In addition, in some embodiments, transfer of energy to the active moiety may result in other chemical reactions within the active moiety, not necessarily leading to the cleavage of a cleavable bond. If present, a releasable moiety may be any suitable moiety that can be released, e.g., during cleavage (including photocleavage).

In some embodiments, the releasable moiety can include a portion of the prodrug or a portion of the cleavable moiety. Different releasable moieties can be used in various embodiments, depending on the application. For example, the releasable moiety may include a drug, a tracer (e.g., a fluorescent or radioactive compound), a caged species, a peptide or protein, a small molecule (e.g., having a molecular weight of less than about 1 kDa or about 2 kDa), or the like. In some embodiments, the exact form of the releasable moiety is not critical, e.g., if it is attached through a cleavable bond of a cleavable moiety that itself is cleaved as discussed above; cleavage of the cleavable bond may thereby cause separation of the releasable moiety, regardless of the exact composition of the releasable moiety.

As non-limiting examples, a releasable moiety is or comprises a drug, such as an anti-cancer drug selected from the group consisting of chlorambucil (CAB), vadimezan, indomethacin, naproxen, ibuprofen, benzyloxycinnamic acid, tetracaine, dopamine, tyramine and homoveratrylamine.

In some embodiments, the prodrug is represented by any one of the following structures:

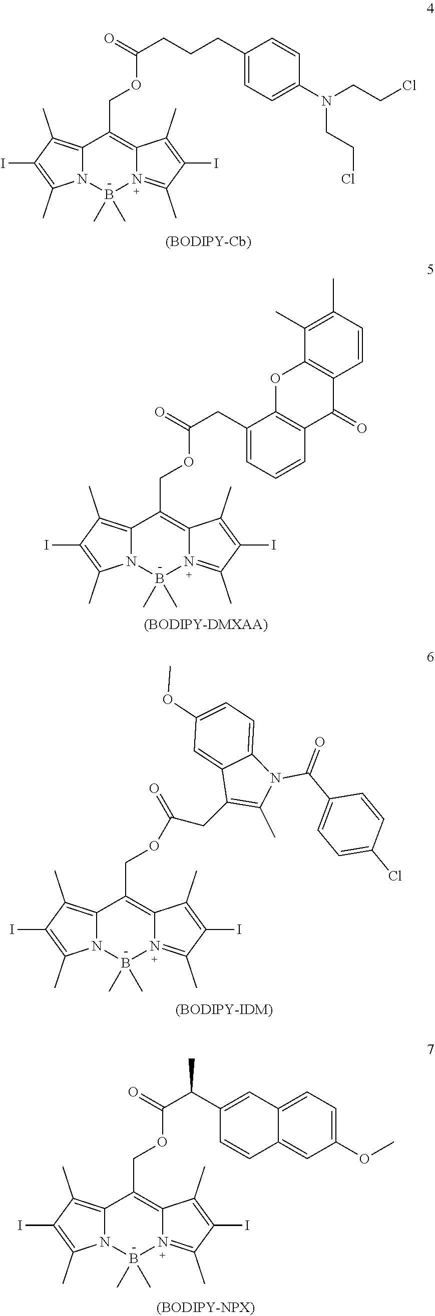

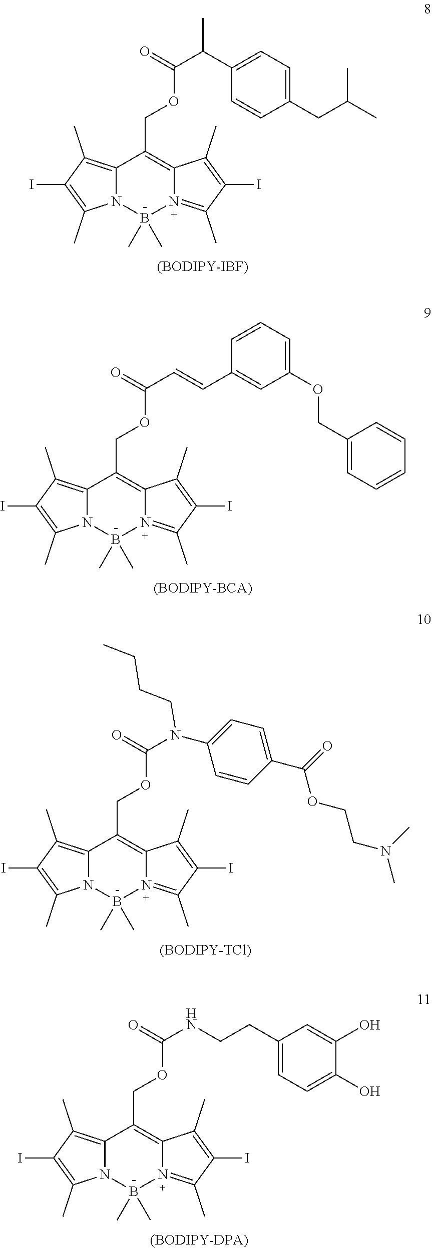

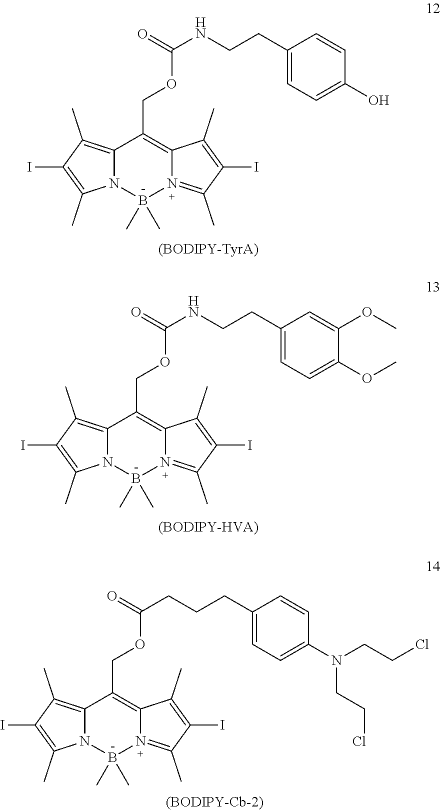

In some embodiments, the present disclosure provides a compound represented by any one of the following structures:

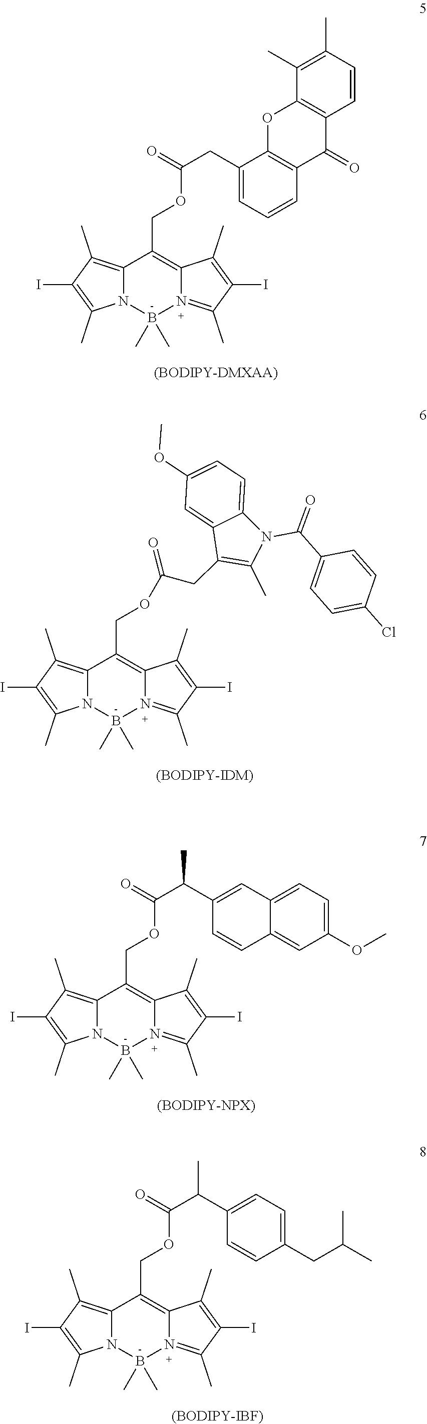

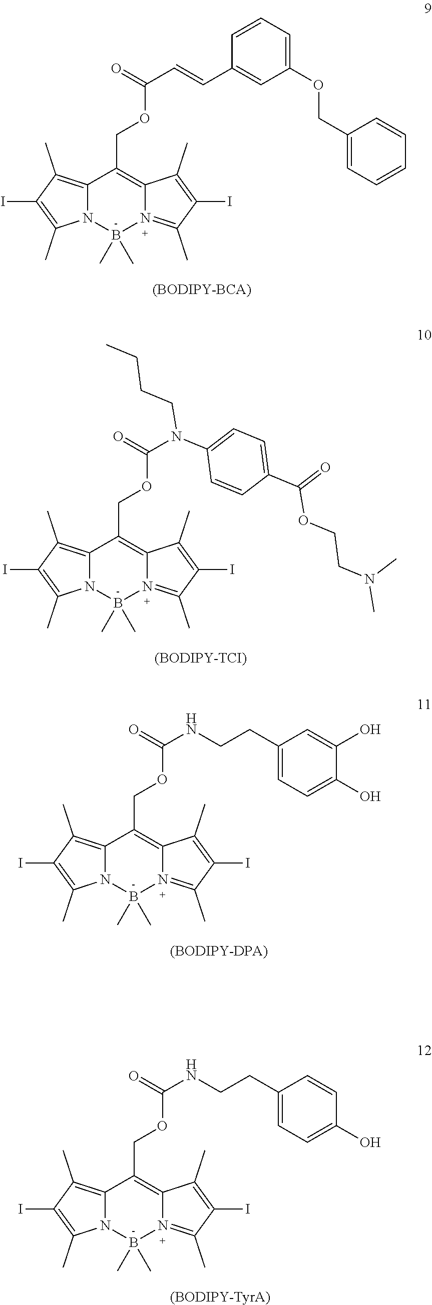

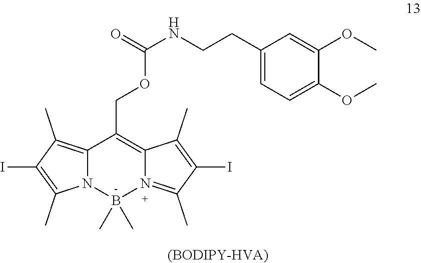

In some embodiments, the compound can be a prodrug comprising a cleavable moiety and a releasable moiety.

In some embodiments, the present disclose provides a compound represented by the structure of formula 5. In some embodiments, the present disclose provides a compound represented by the structure of formula 6. In some embodiments, the present disclose provides a compound represented by the structure of formula 7. In some embodiments, the present disclose provides a compound represented by the structure of formula 8. In some embodiments, the present disclose provides a compound represented by the structure of formula 9. In some embodiments, the present disclose provides a compound represented by the structure of formula 10. In some embodiments, the present disclose provides a compound represented by the structure of formula 11. In some embodiments, the present disclose provides a compound represented by the structure of formula 12. In some embodiments, the present disclose provides a compound represented by the structure of formula 13.

In other embodiments, the releasable moiety can include an anti-angiogenesis drug, such as TNP-470 or Combretastatin A4. In another set of embodiments, the releasable moiety may include an anti-inflammatory drug, such as dexamethasone. In yet another set of embodiments, the releasable moiety includes an anticancer drug and/or a chemotherapy drug, such as chlorambucil, doxorubicin, topotecan, or verteporfin. In yet another set of embodiments, the releasable moiety may include fluorescent proteins, such as GFP or YFP. In still another set of embodiments, the releasable moiety can include fluorescent compounds, such as fluorescein, rhodamine, or calcein. In still another set of embodiments, the releasable moiety includes a peptide or a protein, such as an RGD peptide. In another set of embodiments, the releasable moiety may include a radioactive atom.

### 5.1.3 Carrier

In some embodiments, the photosensitizer, the active moiety (e.g., a cleavable moiety), and/or the releasable moiety (if present) are contained within a suitable carrier material. The carrier material may hold some or all of these in close proximity to each other (e.g., as discussed above). In some embodiments, the carrier material may create an environment favorable for compounds such as those discussed herein to be fluorescent. For example, the carrier material may create an aqueous environment, a hydrophobic environment, a polar or non-polar environment, etc. In some embodiments, the carrier material creates an environment that repels water.

In one set of embodiments, the carrier material is formed from a polymer. Any suitable polymer can be used. Examples of polymers include, but are not limited to, polylactic acid, polyglycolic acid, polyethylene oxide, polystyrene, polyethylene, polypropylene, etc. In some embodiments, the polymer may be biodegradable or biocompatible, e.g., for use in various medical or biological applications. In some embodiments, more than one polymer can be used, and the polymers may be physically blended together and/or chemically combined, e.g., as in a copolymer. As a non-limiting example, the carrier material may include a copolymer such as poly(D,L-lactic acid)-poly(ethylene oxide).

However, it should be understood that the carrier material needs not be limited to polymeric materials. For example, in other embodiments, the carrier material can include silica, ceramics, or other materials.

The carrier material can be present in any suitable form. For example, the carrier material can be present as a film, as a block of material, as particles, as a micelle, or the like. In some embodiments, components such as the photosensitizer, the active moiety, and/or the releasable moiety may be added or chemically conjugated to the carrier material during and/or after formation of the carrier material. The carrier material can be formed using any suitable techniques; for example, techniques for producing polymers, silica gels, ceramics, etc. are known to those of ordinary skill in the art.

If the carrier material is present as particles, the particles may be spherical or nonspherical, and may have any suitable diameter. For instance, the particles may have an average diameter of less than about 1 mm, less than about 500 micrometers, less than about 300 micrometers, less than about 100 micrometers, less than about 50 micrometers, less than about 30 micrometers, less than about 10 micrometers, less than about 5 micrometers, less than about 3 micrometers, less than about 1 micrometer, less than about 500 nm, less than about 300 nm, less than about 100 nm, less than about 50 nm, less than about 30 nm, less than about 10 nm, etc. The average diameter of a nonspherical particle may be taken as the volume of a perfect sphere having the same volume of the particle. If the carrier material is present as a film, the film can have any cross-sectional thickness. For example, the film may have an average thickness of less than about 1 mm, less than about 500 micrometers, less than about 300 micrometers, less than about 100 micrometers, less than about 50 micrometers, less than about 30 micrometers, less than about 10 micrometers, less than about 5 micrometers, less than about 3 micrometers, less than about 1 micrometer, less than about 500 nm, less than about 300 nm, less than about 100 nm, less than about 50 nm, less than about 30 nm, less than about 10 nm, etc.

The carrier material may also comprise one or more polymeric micelles. The polymer micelles may have any suitable average diameter. For example, the micelles can have an average diameter of less than about 1 mm, less than about 500 micrometers, less than about 300 micrometers, less than about 100 micrometers, less than about 50 micrometers, less than about 30 micrometers, less than about 10 micrometers, less than about 5 micrometers, less than about 3 micrometers, less than about 1 micrometer, less than about 500 nm, less than about 300 nm, less than about 100 nm, less than about 50 nm, less than about 30 nm, less than about 10 nm, etc.

### 5.1.4 Therapeutic Methods

As mentioned, compositions such as those discussed herein may be used in a wide variety of applications, including biological and medical applications, as well as non-biological or non-medical applications. As a non-limiting example, in one set of embodiments, a composition as discussed herein may be applied to a subject. The subject may be human or non-human. For example, the subject may be a rat, mouse, rabbit, goat, cat, dog, or the like. The composition can also be applied to any suitable sample, e.g., a biological sample, a physical sample, a chemical sample, or the like.

Light may be applied to the composition to cause release of the releasable moiety, if present. The light may be monochromatic light (e.g., laser or coherent light), or the light may be nonmonochromatic or noncoherent in some embodiments. The light may have any suitable frequency, e.g., including the frequencies discussed herein, such as near infrared (NIR) wavelength.

In one set of embodiments, the composition is applied to a subject to treat a tumor.

The composition may be applied directly to the tumor, and/or applied systemically to the body of the subject such that at least some of the composition is able to travel to the tumor (e.g., via the blood) such that light can be applied to the tumor (or portion thereof), e.g., to cause release of a releasable moiety for diagnosing and/or treating the tumor. The composition can include, for example, an anti-angiogenesis drug, an anti-inflammatory drug, a radioactive species, an anticancer drug and/or a chemotherapy drug, and light may be applied to the tumor to cause release. Such application may be targeted, e.g., by applying light directly to the tumor (or at least a portion thereof); thus, release elsewhere within the subject may be minimized by not applying light to other places. In such a fashion, release of a drug (or other suitable release moiety) may be controlled or localized at or near the tumor by applying light directly to the tumor (or portion thereof), or at least proximate the tumor. In some embodiments, more than one composition may be present.

Other portions of a subject may also be treated in various embodiments. For instance, the composition may be applied directly to a specific location within the subject, or applied systemically to the subject such that at least some of the composition is able to travel to a location where light is to be applied. For instance, the composition may be applied to the skin, eye, body cavity (or to the blood) and light applied to a portion of the skin, eye, body cavity or the blood to cause local release of a releasable moiety.

In various aspects, the compositions described herein can be administered by any suitable method, e.g., contained in a solution or suspension, such as inhalation solutions, local instillations, eye drops, intranasal introductions, an ointment for epicutaneous applications, intravenous solutions, injection solutions (e.g., subcutaneous, or intravenous), or suppositories. In one set of embodiments, the composition is introduced parenterally or topically. For instance, the composition may be contained within a cream, gel, or ointment applied to the skin. In some embodiments, the composition can be applied one or more times a day, by one or more administrations per day, by fewer than one time per day, or by continuous administration, etc., until a desired therapeutic effect is achieved.

As mentioned, certain aspects of the present invention provide methods of administering any composition of the present disclosure to a subject. When administered, the compositions of the invention are applied in a therapeutically effective, pharmaceutically acceptable amount as a pharmaceutically acceptable formulation. As used herein, the term “pharmaceutically acceptable” is given its ordinary meaning. Pharmaceutically acceptable compositions are generally compatible with other materials of the formulation and are not generally deleterious to the subject. Any of the compositions of the present invention may be administered to the subject in a therapeutically effective dose. A “therapeutically effective” amount as used herein means that amount necessary to delay the onset of, inhibit the progression of, halt altogether the onset or progression of, diagnose a particular condition being treated, or otherwise achieve a medically desirable result. When administered to a subject, effective amounts will depend on the particular condition being treated and the desired outcome. A therapeutically effective dose may be determined by those of ordinary skill in the art, for instance, employing factors such as those further described below and using no more than routine experimentation.

Any medically acceptable method may be used to administer the composition to the subject. The administration may be localized (i.e., to a particular region, physiological system, tissue, organ, or cell type) or systemic, depending on the condition to be treated. For example, the composition may be administered orally, vaginally, rectally, buccally, pulmonary, topically, nasally, transdermally, through parenteral injection or implantation, via surgical administration, or any other methods of administration. Examples of parenteral modalities that can be used with the invention include intravenous, intradermal, subcutaneous, intracavity, intramuscular, intraperitoneal, epidural, or intrathecal. Examples of implantation modalities include any implantable or injectable drug delivery systems. Use of an implant may be particularly suitable in some embodiments of the invention. The implant containing the composition may be constructed and arranged to remain within the body for at least 2-4 hours, 4-12 hours, 12-24 hours, 24-48 hours, 1-7 days, 7-15 days, for at least 30 or 45 days, and preferably at least 60 or 90 days, or even longer in some cases. Long-term release implants are well known to those of ordinary skill in the art.

In certain embodiments of the invention, a composition can be combined with a suitable pharmaceutically acceptable carrier, for example, as incorporated into a liposome, incorporated into a polymer release system, or suspended in a liquid, e.g., in a dissolved form, or a colloidal form, or a micellular form. In general, pharmaceutically acceptable carriers suitable for use in the invention are well-known to those of ordinary skill in the art. A pharmaceutically acceptable carrier may include non-toxic material that does not significantly interfere with the effectiveness of the biological activity of the active compound(s) to be administered, but is used as a formulation ingredient, for example, to stabilize or protect the active compound(s) within the composition before use. The carrier may be organic or inorganic, and may be natural or synthetic, with which one or more active compounds of the invention are combined to facilitate the application of the composition. The carrier may be either soluble or insoluble, depending on the application.

A kit of the invention may, in some embodiments, include instructions in any form that are provided in connection with the compositions of the invention in such a manner that one of ordinary skill in the art would recognize that the instructions are to be associated with the compositions of the invention. For instance, the instructions may include instructions for the use, modification, mixing, diluting, preserving, administering, assembly, storage, packaging, and/or preparation of the composition and/or other compositions associated with the kit. In some embodiments, the instructions may also include instructions for the delivery and/or administration of the compositions.

The following examples are intended to illustrate certain embodiments of the present invention, but do not exemplify the full scope of the invention.

### 6. EXAMPLES

### 6.1 Preparation and Characterizations of Osmium-Based STPS and BODIPY-Cb Prodrug

A BODIPY-chlorambucil prodrug (BODIPY-Cb) is created that can release chlorambucil as an anti-cancer agent for photoactivated therapy under green light irradiation.¹⁷ As STPS, Osmium (II) complexes exhibit strong singlet-to-triplet absorption, which enables direct excitation from its ground state to T₁ state.¹⁹ Here, an osmium-containing photosensitizer, Os (II) bromophenyl terpyridine complex (Os(bptpy)₂²⁺), was synthesized) and characterized (referred to Supporting Information, Scheme S2).

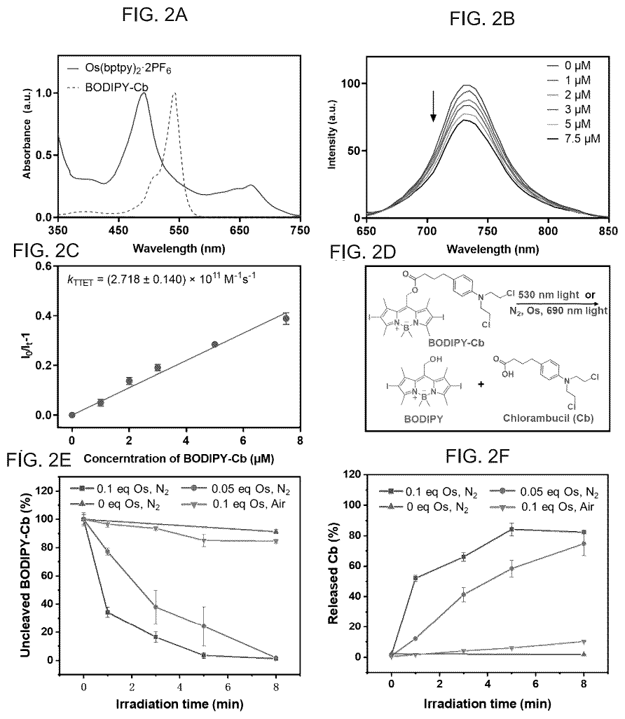

As shown in the UV-Vis spectrum, the BODIPY-Cb prodrug exhibits a single absorption peak in the visible-light area (peaked at 543 nm, ε=8.94×10⁴ M⁻¹ cm⁻¹), indicating green light excitation. The Os(bptpy)₂²⁺ exhibits broad peaks in the visible-to-NIR area (FIG. 2A). The absorption peak at about 492 nm is identified as the singlet metal-to-ligand charge transfer (¹MLCT) absorption, also termed as singlet-singlet (S-S) absorption. Notably, the peak in the far red-NIR area (peaked at 688 nm, ε=2.66×10⁴ M⁻¹ cm⁻¹), is identified as the triplet metal-to-ligand charge transfer (³MLCT) absorption (also termed as S-T absorption), demonstrating that it can be excited by NIR light. The two twistable bromophenyl groups of the bromophenyl terpyridine ligands extend the S-T absorption from 650 nm to 750 nm with increased absorption coefficient. More detailed photophysical properties of BODIPY-Cb and Os(bptpy)₂²⁺ and BODIPY-Cb were recorded in Table 1 below, including the Supporting Information.

TABLE 1
Photophysical properties of BODIPY-Cb and Os(bptpy)₂•2PF₆.

| | λ_{abs, max} | E | λ_{em, max} | T₁ lifetime | Energy level (eV) | |
|---|---|---|---|---|---|---|
| Compound | (nm)^{a} | (M⁻¹ cm⁻¹) | (nm) | (μs) | S₁ state | T₁ state |
| BODIPY-Cb | 543 | 8.94 × 10⁴ | 570^{b} | 21.82^{e} | 2.18^{f} | 1.52^{g} |
| Os(bptpy)₂•2PF₆ | 491 (S₀-S₁) | 7.35 × 10⁴ | 498^{c} | 0.20^{f} | 2.56^{h} | 1.69ⁱ |
| | 688 (S₀-T₁) | 2.66 × 10⁴ | 734^{d} | | | |

^{a}In dichloromethane solution with 2% acetone, 10 μM
^{b}λₑₓ = 543 nm;
^{c}λₑₓ = 491 nm;
^{d}λₑₓ = 688 nm;
^{e,f,g}In toluene, calculated by TD-DFT, referred to previous studies ^{37, 38};
^{h}Calculated by maximum S₁ emission wavelength (λₑₓ), E = 1240/λ_{em, S1}; and
ⁱCalculated maximum T₁ emission wavelength (λₑₘ), E = 1240/λ_{em, T1}.

Based on the phosphorescence emission, the T₁ energy level of Os(bptpy)₂²⁺ was determined as 1.69 eV, which is consistent of density functional theory (DFT) calculation (Table 1).²⁰ Thus, low-energy NIR photons can be used to activate it. BODIPY-Cb exhibited high singlet excitation state (S₁=2.18 eV) and low triplet state (T₁=1.52 eV).¹⁷ Based on the energy levels, this molecule pair, Os(bptpy)₂²⁺ and BODIPY-Cb, satisfies the energy requirement for TTET (T₁ (PS)>T₁ (PD)) and upconversion-like process (T₁ (PS)<S₁ (PD)) (FIG. 1D). After applying NIR light and activating Os(bptpy)₂²⁺ to the triplet state (1.69 eV), the photon energy can be transferred to the triplet state of BODIPY prodrug (1.52 eV) and trigger its photolysis.

### 6.2 TTET Between Os(Bptpy)₂²⁺ and BODIPY-Cb Prodrug

Stern-Volmer phosphorescence quenching assay was used to verify the TTET from Os(bptpy)₂²⁺ to BODIPY-Cb. The phosphorescence of Os(bptpy)₂²⁺ was found to be quenched by titrating BODIPY-Cb prodrug into its N₂-saturated toluene solution. It was observed that the phosphorescence of Os(bptpy)₂²⁺ decreased while increasing the BODIPY-Cb concentration, which verifies the energy transfer from ³Os(bptpy)₂²⁺ to ³BODIPY1-Cb*(FIGS. 2, B and C). The TTET rate constant (k_{TTET}) was calculated as (2.72±0.14)×10¹¹ M⁻¹ s⁻¹, indicating efficient energy transfer from T₁ state of Os(bptpy)₂²⁺ to T₁ state of BODIPY-Cb.

### 6.3 Photolysis of BODIPY-Based Prodrugs Via Upconversion-Like Process

The photolysis of BODIPY-Cb (FIG. 2D) was studied by irradiating solutions with 530 nm or 690 nm lamps and analysing the products by high-performance liquid chromatography (HPLC). Table 2 below shows data for the HPLC method used for photolysis study.

TABLE 2
HPLC method for photolysis study.

| Time | Acetonitrile | H₂O |
|---|---|---|
| (min) | (0.1 % TFA, v/v) | (0.1 % TFA, v/v) |
| 0 | 20% | 80% |
| 10 | 40% | 60% |
| 15 | 80% | 20% |
| 18 | 100% | 0% |
| 30 | 100% | 0% |

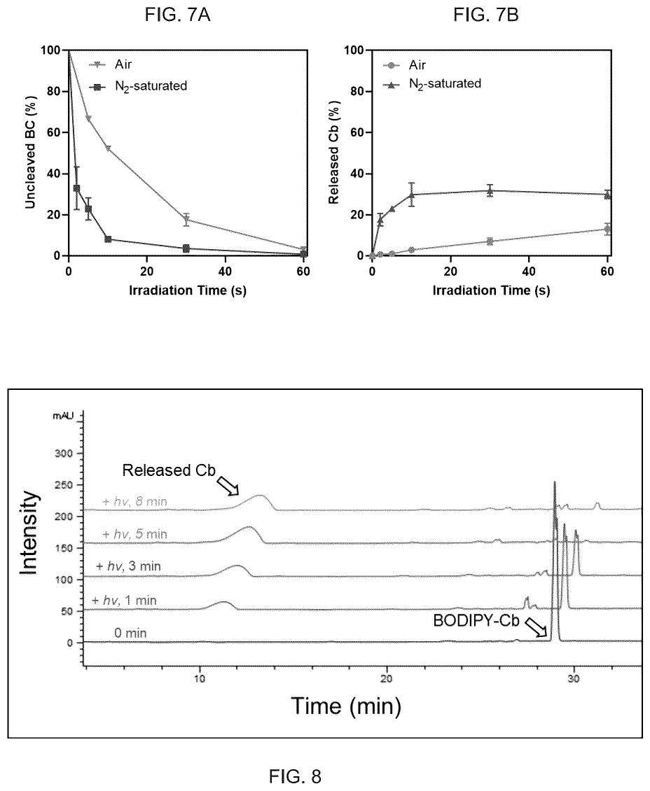

It was observed that BODIPY-Cb can be cleaved under 530 nm green light, since the high-energy photons can directly activate the prodrug to the S1 state of BODIPY1-Cb, followed by the cleavage relaxation and generation of free drug (FIG. 7). Notably, the photocleavage was retarded in air-saturated solution, indicated that the cleavage relaxation can occur from the T₁ state which was quenched by the oxygen. In the existence of Os(bptpy)₂²⁺, both decomposition of the prodrug and generation of free Cb were observed upon 690 nm light irradiation in N₂-saturated solution (FIG. 2E-F, FIG. 8). It can be explained by the TTET process, where BODIPY-Cb was promoted to T₁ state after accepting the energy from T₁ of Os(bptpy)₂²⁺. Also, the generation of free Cb was accelerated while increasing the molar ratio of Os(bptpy)₂²⁺ in the solution. In the existence of 0.1 equiv. of Os(bptpy)₂²⁺, BODIPY-Cb decomposed nearly completely (96.74±1.26%) upon the light irradiation at 100 mW/cm² for 5 min and generated free Cb at a relative high yield of 84.17±4.21%. It should be noted that the yield of free drug is much higher than that of BODIPY-Cb photolysis with shorter wavelength light, including the direct photolysis by green-light irradiation (max. yield at 31.71%) (FIG. 7) and the one-photon upconversion-like photolysis with platinum photosensitizer by red-light irradiation (max. yield at 41.74%).¹⁷ This result showcased the advantages of utilizing low-energy NIR photons and simplifying energy transfer processes, which reduced photodamage of the prodrugs and unexpected relaxation of the excited states. High photolysis yield of the free drugs may lead to better therapeutic efficacy of the prodrug upon light irradiation. Besides, as expected, the decomposition of BODIPY-Cb as well as the generation of free Cb were mostly retarded in the air-saturated solution since the T₁ states were quenched by the oxygen molecules (green lines, FIG. 2E-F). For details, the quantum yield of photolysis (Φₚ), quantum yield of drug release (Φᵣ) and the cross sections are recorded in Table 3 below.

TABLE S3
Quantum yields and cross sections of BODIPY-Cb prodrug in different conditions.

| | | | Quantum | | | |
|---|---|---|---|---|---|---|
| | | | yield of | Cross | Quantum | Cross |
| | | | prodrug | section | yield of drug | section |
| | λₑₓ | | photolysis | Φₚε(λₑₓ) | release | Φᵣε(λₑₓ) |
| Compound | (nm) | Conditions | Φₚ (%) | (M⁻¹cm⁻¹) | Φᵣ (%) | (M⁻¹cm⁻¹) |
| BODIPY-Cb | 530 | N₂ | 23.89 ± 3.05 | 16017.11 ± 2044.88 | 6.73 ± 2.12 | 6016.62 ± 1895.28 |
| | | Air | 3.42 ± 1.09 | 2292.95 ± 730.79 | (2.56 ± 0.37) × | 171.63 ± 24.81 |
| | | | | | 10⁻¹ | |
| | 690 | 0.1 eq Os, | 0.84 ± 0.01 | 222.80 ± 2.90 | 0.73 ± 0.04 | 193.85 ± 9.70 |
| | | N₂ | | | | |
| | | 0.1 eq Os, | (7.03 ± 4.18) × | 1.87 ± 1.11 | (1.86 ± 0.4) × | 0.49 ± 0.11 |
| | | Air | 10⁻³ | | 10⁻³ | |

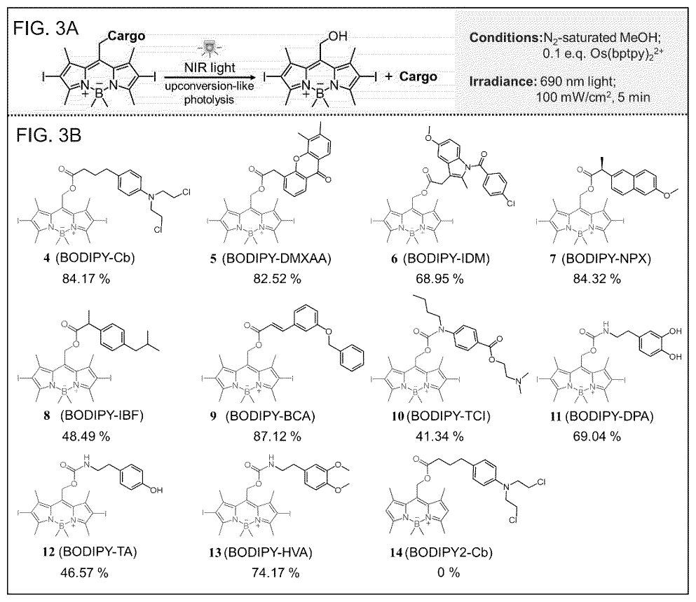

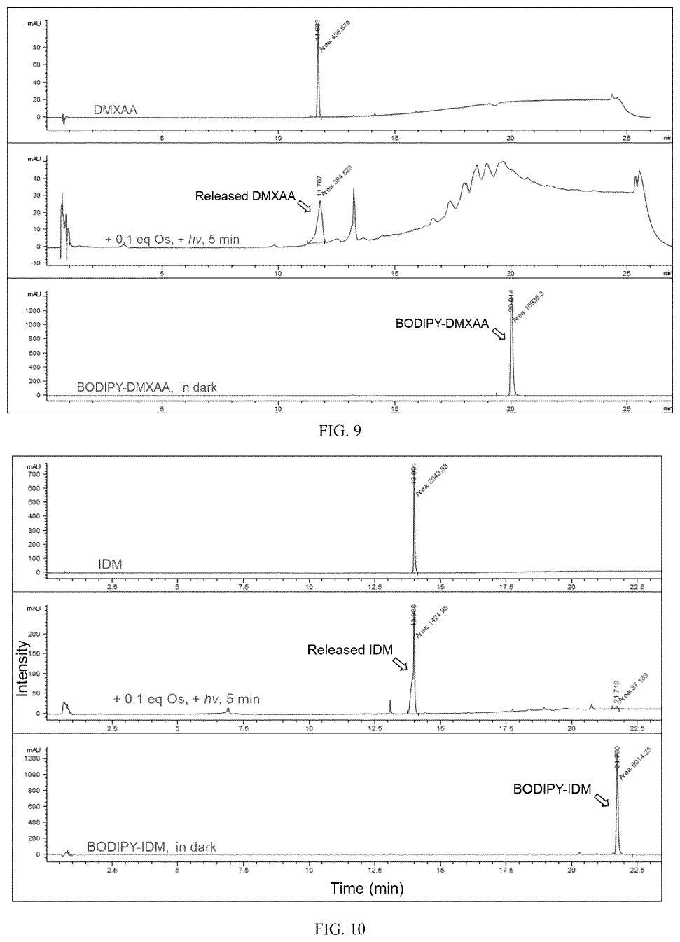

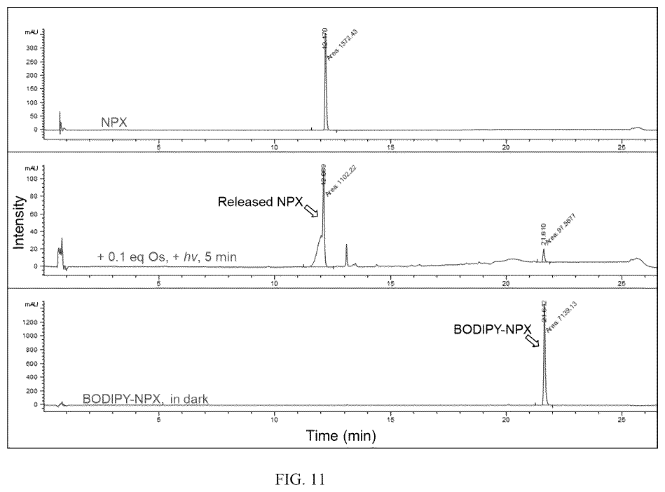

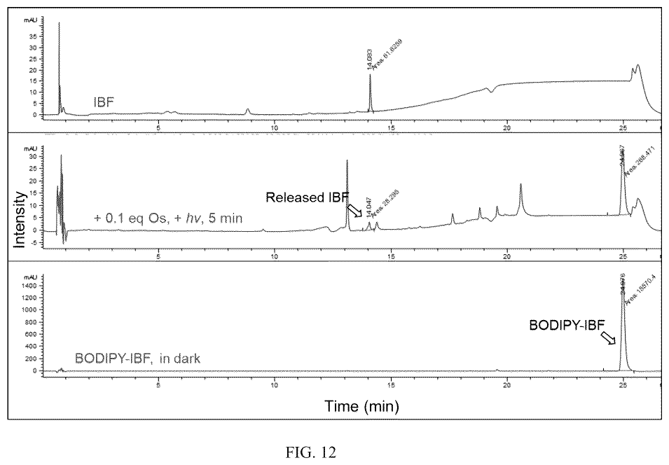

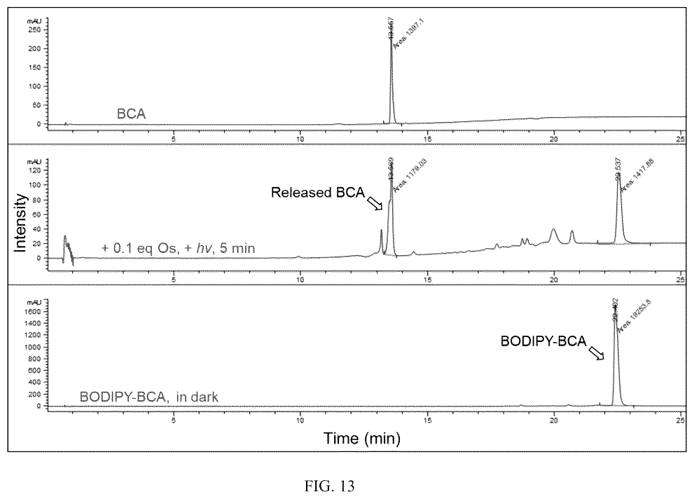

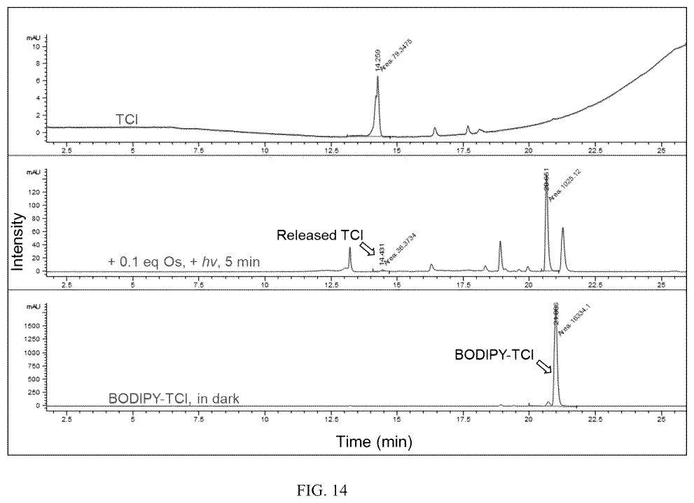

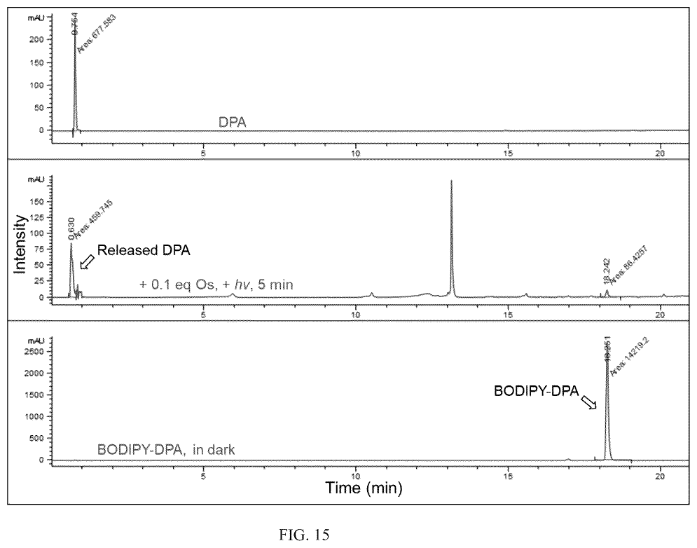

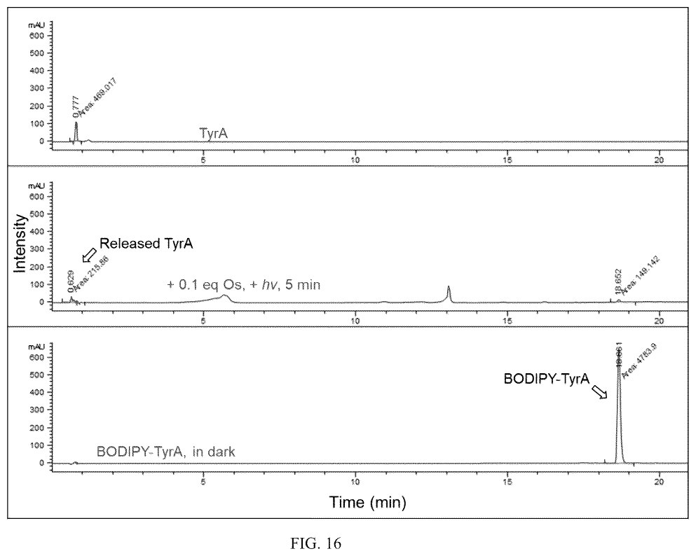

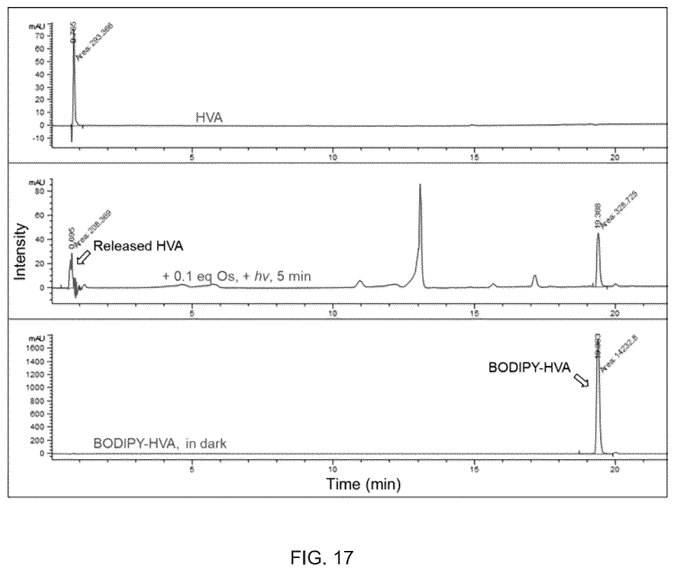

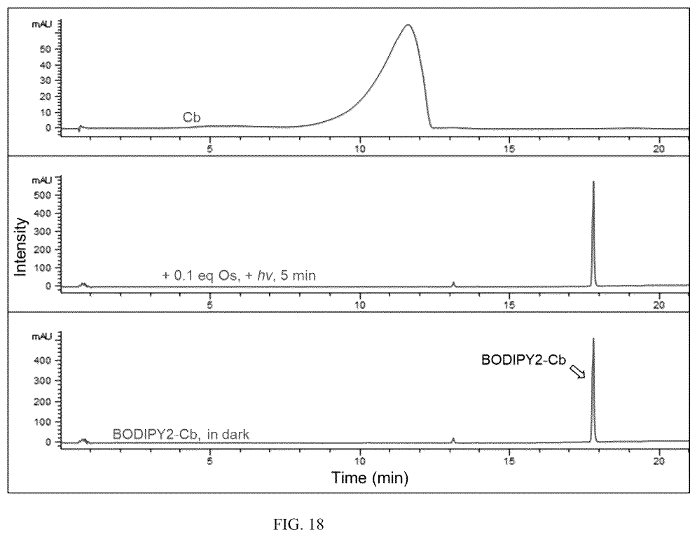

Prodrug photolysis has been emerged as a spatiotemporally controllable process for optochemical control of biological processes. The related studies largely depend on the precise deprotection of bioactive molecules under light illumination.²¹⁻²³ Encouraged by the NIR light-triggered photolysis of BODIPY-Cb, we expanded this concept as NIR light-triggered release of different bioactive molecules, including a) anti-cancer drugs, e.g., chlorambucil (Cb) and vadimezan (DMXAA); b) anti-inflammation drugs, e.g., indomethacin (IDM), naproxen (NPX), ibuprofen (IBF), and benzyloxy cinnamic acid (BCA); c) anaesthesia agent, e.g., tetracaine (TCI); d) biogenic amines, e.g., dopamine (DPA), tyramine (TyrA) and homoveratrylamine (HVA) (FIG. 3). BODIPY photocage was conjugated with the free drug molecules to form photoactivable prodrugs with photolabile linkages, including ester and carbamate. The light illumination was performed with a NIR lamp, of which the parameters were 100 mW/cm², 690 nm for 5 min, in a N₂-saturated solution containing 0.1 equiv. of Os(bptpy)₂²⁺ and 1 equiv. of prodrugs. For BODIPY-chlorambucil (BODIPY-Cb, compound 4), the photolytic yield was measured as 84.17±4.21%, as mentioned above. For BODIPY-vadimezan (BODIPY-DMXAA, compound 5), the prodrug was completely consumed with a yield of free DMXAA at 82.52±7.22% (FIG. 9). For BODIPY-indomethacin (BODIPY-IDM, compound 6), BODIPY-naproxen (BODIPY-NPX, compound 7), BODIPY-ibuprofen (BODIPY-IBF, compound 8) and BODIPY-benzyloxycinnamic acid (BODIPY-BCA, compound 9), the photolytic yields were 68.95±4.69%, 84.32±5.79%, 48.49±4.69% and 87.12±3.17%, respectively (FIG. 10-13). It should be noted that the above prodrugs (compound 4-9) were fabricated with photolabile ester bonds by conjugating BODIPY photocage and the drug molecules with carboxylic groups. In addition, the photocage was conjugated with drug molecules with amine groups to produce prodrugs with photolabile carbamate bonds. As a result, BODIPY-tetracaine (BODIPY-TCI, compound 10) exhibited photolytic yield at 41.34±4.61% after 5-min NIR-light irradiation (FIG. 14). For BODIPY-dopamine (BODIPY-DPA, compound 11), BODIPY-tyramine (BODIPY-TyrA, compound 12) and BODIPY-homoveratrylamine (BODIPY-HVA, compound 13), the photolytic yields were 69.04±1.99%, 46.57±2.37%, and 74.17±3.97%, respectively (FIG. 15-18). Furthermore, BODIPY2-Cb prodrug (compound 14), whose structure is similar to that of BODIPY-Cb but without iodine insertion at the 2- and 6-positions, showed no photolytic yield upon NIR light in the present of Os(bptpy)₂²⁺, which can be explained by that the lack of iodine atoms leads to inefficient ISC efficiency and shortened T₁ lifetime.²⁴ In addition, the quantum yields and cross sections of different prodrugs are listed in Table 4 below.

TABLE 4
Quantum yields and cross sections of BODIPY prodrugs
photolysis under NIR light with λex = 690 nm and conditions
being 0.1 eq Os, N₂.

| | Quantum | Cross | Quantum | |
|---|---|---|---|---|
| | yield of | section | yield of | Cross section |
| | photolysis | Φₚε(λₑₓ)ª | drug release | Φᵣε(λₑₓ)ª |
| Compound | Φp (%) | (M⁻¹ cm⁻¹) | Φr(%) | (M⁻¹ cm⁻¹) |
| BODIPY- | 0.84 ± 0.01 | 222.80 ± 2.90 | 0.73 ± 0.04 | 193.85 ± 9.70 |
| Cb | | | | |
| BODIPY- | 0.87 ± 0.01 | 230.30 ± 1.43 | 0.71 ± 0.06 | 190.05 ± 16.63 |
| DMXAA | | | | |
| BODIPY- | 0.86 ± 0.02 | 229.15 ± 5.23 | 0.60 ± 0.04 | 158.79 ± 10.80 |
| IDM | | | | |
| BODIPY- | 0.85 ± 0.02 | 227.15 ± 4.42 | 0.73 ± 0.05 | 194.19 ± 13.33 |
| NPX | | | | |
| BODIPY- | 0.85 ± 0.04 | 226.34 ± 10.96 | 0.42 ± 0.04 | 111.67 ± 10.81 |
| IBF | | | | |
| BODIPY- | 0.80 ± 0.02 | 213.33 ± 7.55 | 0.75 ± 0.03 | 200.64 ± 7.30 |
| BCA | | | | |
| BODIPY- | 0.81 ± 0.06 | 215.84 ± 15.71 | 0.36 ± 0.04 | 95.21 ± 10.62 |
| TCI | | | | |
| BODIPY- | 0.86 ± 0.01 | 228.90 ± 3.59 | 0.60 ± 0.02 | 159.00 ± 4.58 |
| DPA | | | | |
| BODIPY- | 0.84 ± 0.02 | 223.12 ± 6.28 | 0.40 ± 0.02 | 107.25 ± 5.46 |
| TyrA | | | | |
| BODIPY- | 0.85 ± 0.04 | 224.98 ± 9.83 | 0.64 ± 0.03 | 170.82 ± 9.14 |
| HVA | | | | |

^{a}ε(λₑₓ) here was the absorption coefficient of Os(bptpy)₂ · 2PF₆ at 688 nm (ε = 26600 M⁻¹ cm⁻¹)

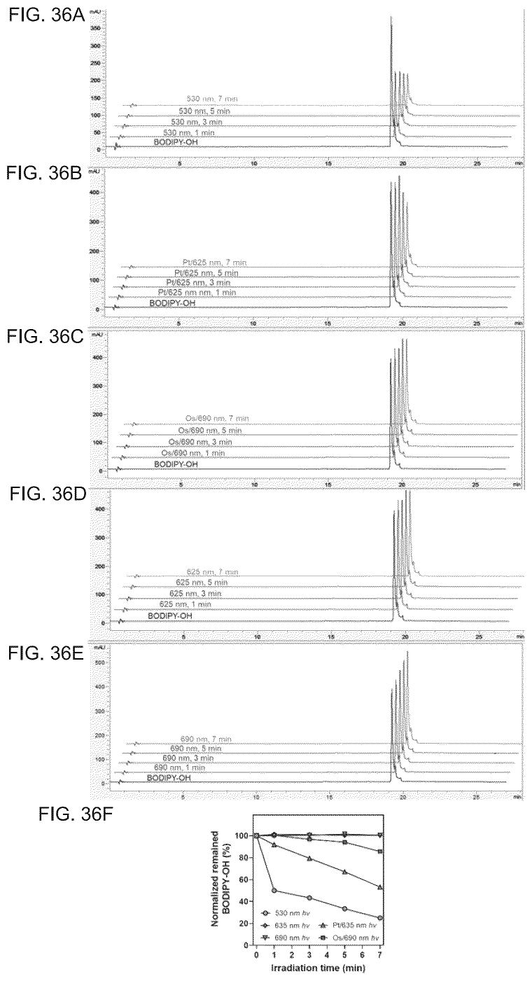

Compared to the disclosure in US2021/0228719, the improvement is the increase of the photolysis yield of the free drugs. In the present disclosure, with 0.1 equiv. of Os(bptpy)₂²⁺, BODIPY-Cb decomposed completely upon the light irradiation at 100 mW/cm² for 5 min and generated free Cb at a relatively high yield of 84.17±4.21%. The yield of free drug is much higher than that of BODIPY-Cb photolysis with shorter wavelength light, including the direct photolysis by green-light irradiation (max. yield at 31.71%) and the upconversion-like photolysis with PtTPBP photosensitizer by red-light irradiation (max. yield at 41.74%) (disclosed in US2021/0228719). This observation can be explained by the reduced photobleaching by using 690 nm NIR light but not the 530 nm green light or 625 nm red light for photolysis. FIG. 36 shows different photobleaching rates of BODIPY-Cb under different light.

### 6.4 Photoactivable Nanosystem for NIR Light-Triggered Drug Release

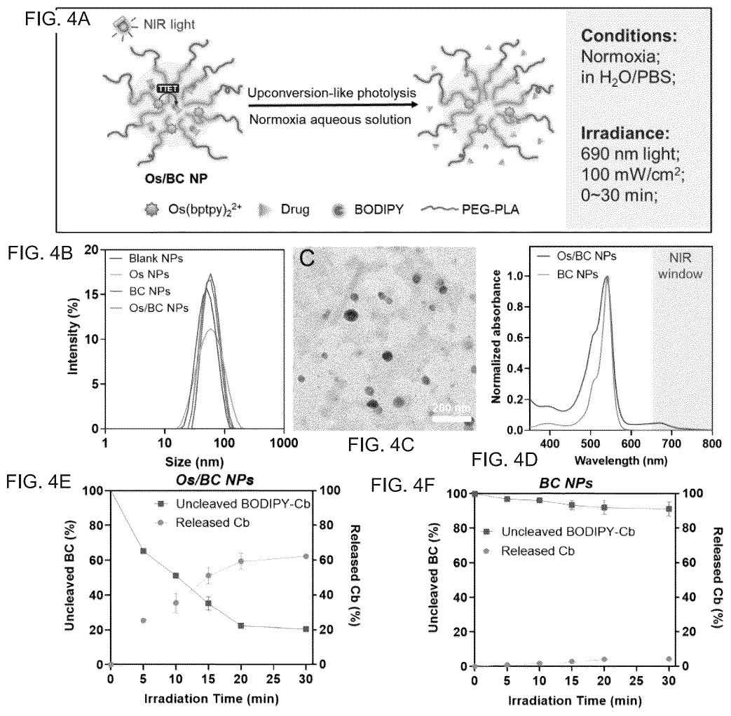

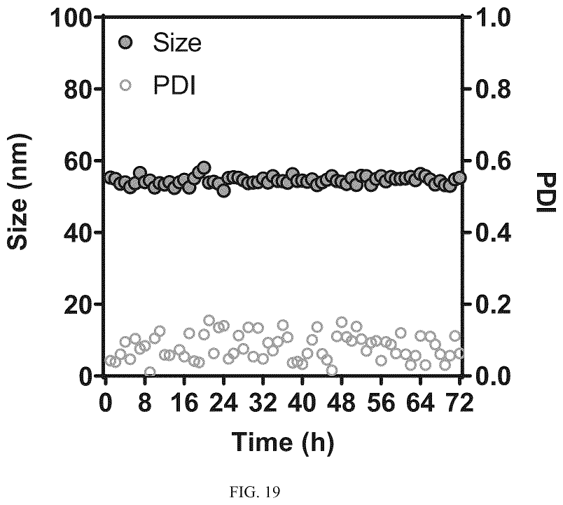

NIR light-triggered prodrug activation was then investigated in biological systems for medical applications. Considering that both the prodrug and photosensitizer are hydrophobic and the energy transfer process is oxygen-sensitive, activating photolabile prodrugs through the upconversion-like mechanism in normoxia aqueous solution would be challenging. Therefore, we loaded the prodrug and photosensitizer in polymeric nanoparticles, which can protect the triplet state from oxygen quenching and thus enable the TTET-based photolysis and drug release in biological environments. An FDA-approved block copolymer, poly(lactic acid)-poly(ethylene glycol) (PLA₅₀₀₀-mPEG₅₀₀₀), was used to fabricate biocompatible and biodegradable nanoparticles (FIG. 4A).²⁵ Dynamic light scattering (DLS) recorded the size of blank nanoparticles (blank NPs), Os(bptpy)₂²⁺-loaded NPs (Os NPs), BODIPY-Cb-loaded NPs (BC NPs) and Os(bptpy)₂²⁺ plus BODIPY-Cb-loaded NPs (Os/BC NPs) at around 50 nm (as shown in Table 5 below), which was also verified by TEM imaging (FIGS. 4B and C). Excellent colloidal stability of the nanoparticles was observed, of which the size remained stable for at least 72 h at 37° C. (FIG. 19). Besides, the absorption spectra of Os/BC NPs displayed peaks in both visible area (540 nm) and NIR area (690 nm), indicated successful encapsulation of Os(bptpy)₂²⁺ and BODIPY-Cb in the nanoparticles (FIG. 4D, Table 6 below).

TABLE 5
Size and PDI of Os NPs, BC NPs and Os/BC NPs.

| | Size (nm) | PDI |
|---|---|---|
| Blank NPs | 47.20 ± 0.35 | 0.10 ± 0.01 |
| Os NPs | 54.58 ± 0.60 | 0.22 ± 0.02 |
| BC NPs | 56.20 ± 2.28 | 0.16 ± 0.03 |
| Os/BC NPs | 53.51 ± 0.70 | 0.13 ± 0.02 |

TABLE 6
Composition of Os/BC NP.

| | Feeding | Weight after | Encap- | |
|---|---|---|---|---|
| | weight | purification | sulation | Loading |
| | (mg) | (mg) | Efficiency | Capacity |
| PLA5k-mPEG5k | 400 | / | / | / |
| Os(tpbpy)2 · 2PF6 | 3.02 | 0.89 | 29.43% | 0.22% |
| BODIPY-Cb | 1.94 | 1.11 | 55.79% | 0.27% |

Light-triggered drug release has attracted many interests for drug delivery and precise disease treatment.²⁶⁻²⁸ The prodrug activation and drug release from of Os/BC NPs were investigated under 690 nm light irradiation at 100 mW/cm². The release of free drug Cb as well as the consumption of BODIPY-Cb were obvious after light irradiation, indicated that the upconversion-like photolysis process took place in the Os/BC NPs which was dispersed in normoxia aqueous solutions. Quantitatively, both the decomposition of prodrug BODIPY-Cb and release of Cb increased along with the irradiation time from 0 to 30 min (FIG. 4E). The drug release percentage was detected as 62.24% after 30-min light irradiation, while 79.52% of BODIPY-Cb was consumed. As compared, the nanoparticles encapsulating only BODIPY-Cb (BC NPs) showed negligible Cb release upon NIR-light irradiation, indicating the prodrug photolysis within the nanoparticles depends on the energy transfer from Os(bptpy)₂²⁺ to BODIPY-Cb (FIG. 4F). In all, these observations confirmed that Os/BC NPs enabled NIR light-triggered drug release in normoxia aqueous solutions.

### 6.5 In Vitro and In Vivo Photoactivated Cancer Therapy

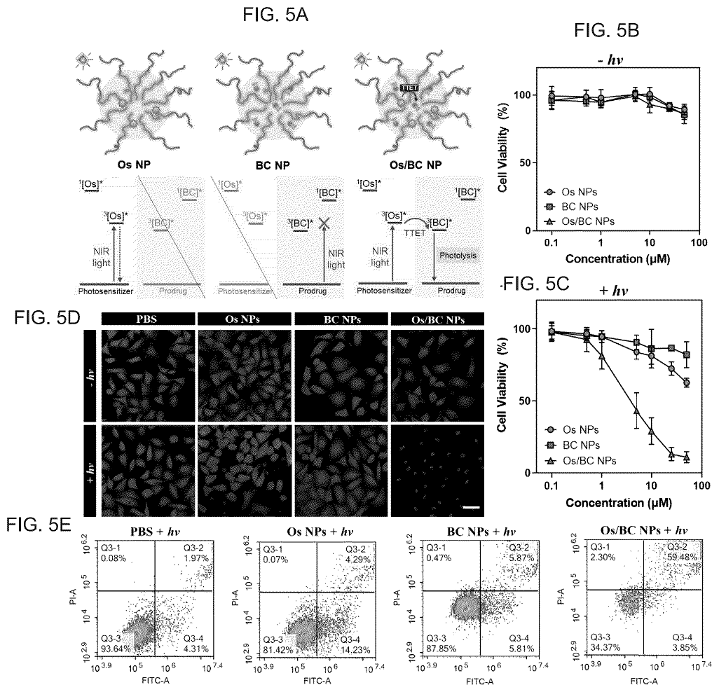

In vitro and in vivo studies of light-controllable cancer treatment with Os/BC NPs were further conducted. Chlorambucil is an FDA-approved anti-tumor drug that has been applied in clinical cancer therapy since 1950s.²⁹ Some chlorambucil prodrugs have been developed, which reduced its systemic side effects by hindering the off-target toxicity and enhanced therapeutic efficacy by precise activation at lesions.^{30, 31} Here, the cytotoxicity of the light-triggered chlorambucil release from the nanoparticles was investigated through the 3-(4,5-dimethylthiazol-2-yl) 2,5-diphenyl tetrazolium bromide (MTT) assay against human cervical cancer (HeLa) cells. It was observed that Os/BC NPs exhibited significant anti-proliferation effects towards HeLa cells upon NIR-light irradiation (690 nm, 100 mW/cm², 30 min), while BC NPs displayed negligible toxicity (FIG. 5C). Os NPs slightly inhibited the cell growth after NIR-light exposure, indicating limited phototoxicity without prodrug activation. Besides, in dark environment, Os NPs, BC NPs and Os/BC NPs all exhibited minimal cytotoxicity (FIG. 5B). For further confirmation, live-dead staining analysis was conducted by Calcein AM/PI co-staining assay. Large proportion of dead cells presenting red fluorescence were observed in the Os/BC NPs plus light-treated group, while other groups did not cause obvious cell death (FIG. 5D). The results coincided well with the cytotoxicity study, demonstrating that the light-triggered prodrug activation and drug release from Os/BC NPs efficiently inhibited the growth of cancer cells.

To further investigate the mechanisms of cell death triggered by prodrug photolysis, HeLa cells treated with Os NPs, BC NPs, and Os/BC NPs followed by NIR-light irradiation were stained with Annexin-V FITC/PI to investigate apoptosis process (FIG. 5E). The results showed that about 63.33% of cells were apoptotic after the treatment with Os/BC NPs (equivalent concentration of BC at 10 μM) and NIR-light irradiation, which was largely dominated by late apoptosis (59.48%). Much less proportion of apoptotic cells were observed in the groups treated with Os NPs and BC NPs. In all, the results confirmed that the cytotoxicity of Os/BC NPs was mainly based on the apoptosis-inducing effect after light-triggered release of Cb.

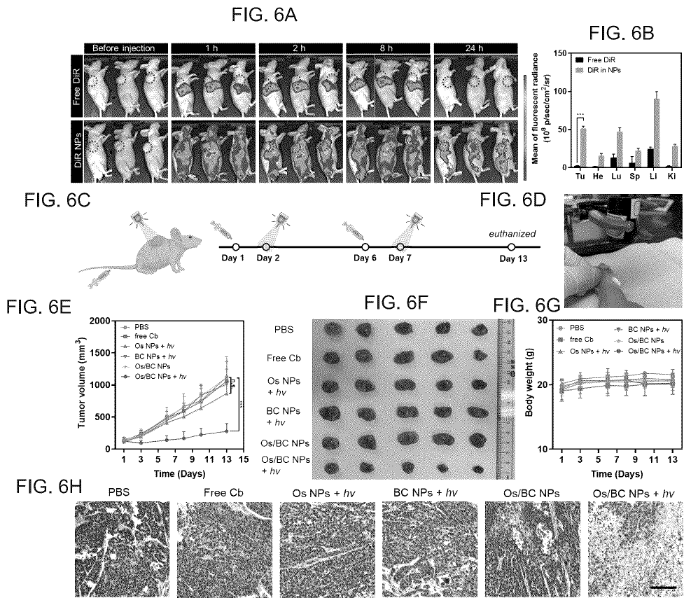

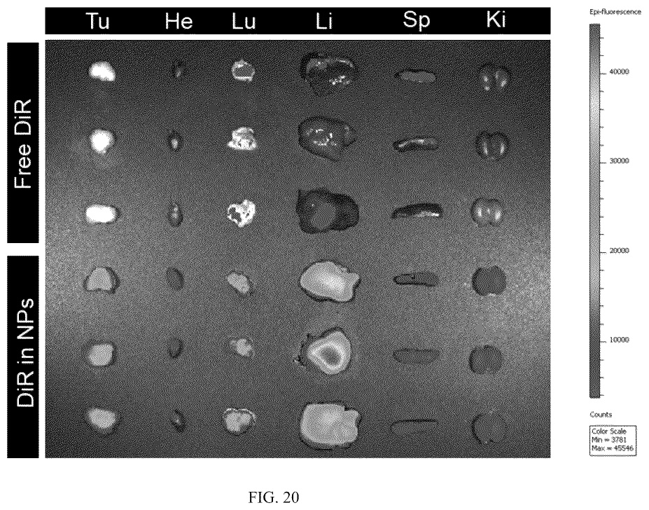

It was reported that PLA-mPEG micellar nanoparticles displayed circulation stability and tumor accumulation ability based on enhanced permeability and retention (EPR) effect in tumors after systemic administration.^{32, 33} For verification, we labelled the nanoparticles with 1,1′-dioctadecyl-3,3,3′,3′-tetramethylindotricarbocyanine iodide (DiR) dye and examined the biodistribution of DiR-loaded nanoparticles (DiR NPs) after intravenous injection into HeLa tumor-bearing mice. Based on fluorescence observation with an in vivo imaging system (IVIS), it was found that the nanoparticles exhibited both longer circulation time and tumor-accumulation ability as compared to free DiR (FIG. 6A). The fluorescence signal representing DiR NPs was obviously enhanced in tumor areas with the increase of time from 0 h to 24 h, while the free dye was completely metabolized within the first 8 h. The tumors and major organs were excised for ex vivo fluorescence imaging 24-h post injection. As a result, the nanoparticles exhibited preferential accumulation and retention capability in tumors (FIG. 6B and FIG. 20). It should be noted that the prodrug can be selectively activated in tumors by NIR light while those nanoparticles in the normal tissues and organs will not be activated, which can alleviate the side effects of chemotherapy.

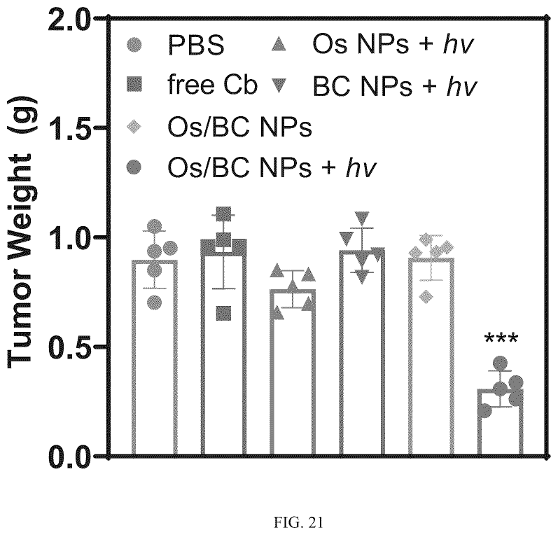

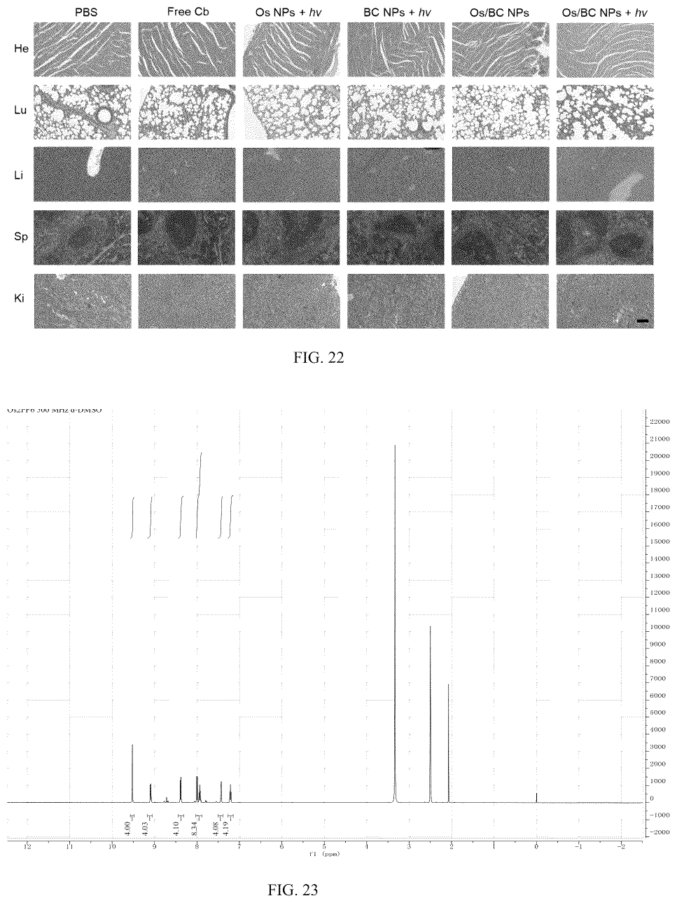

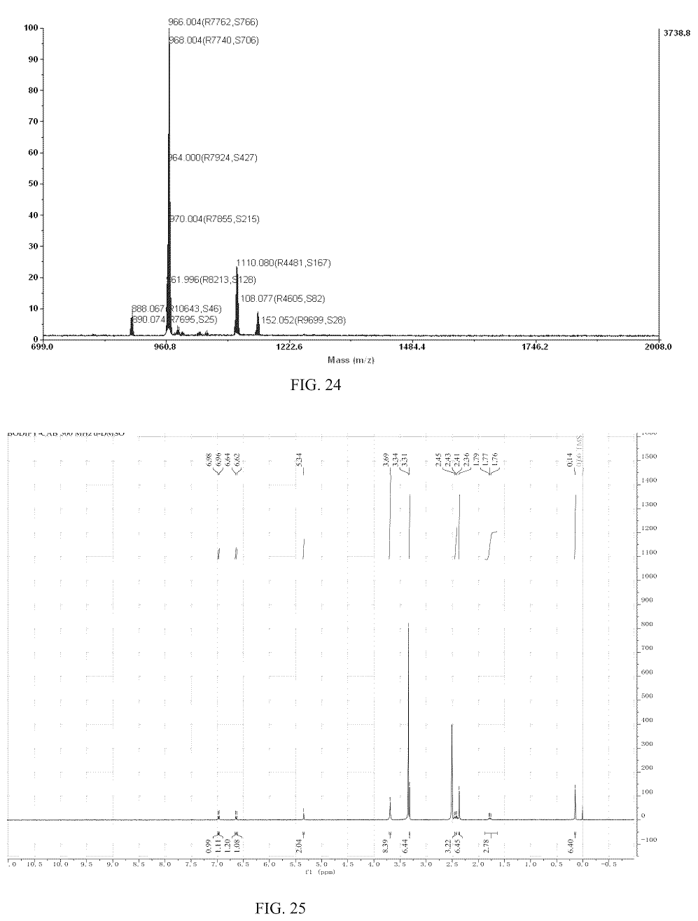

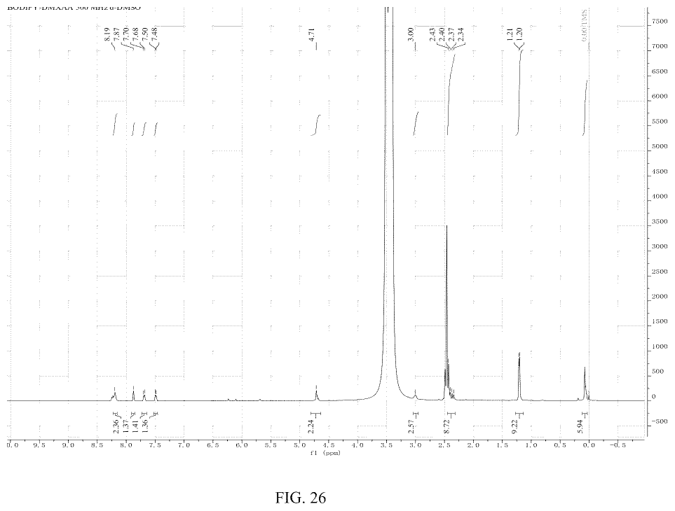

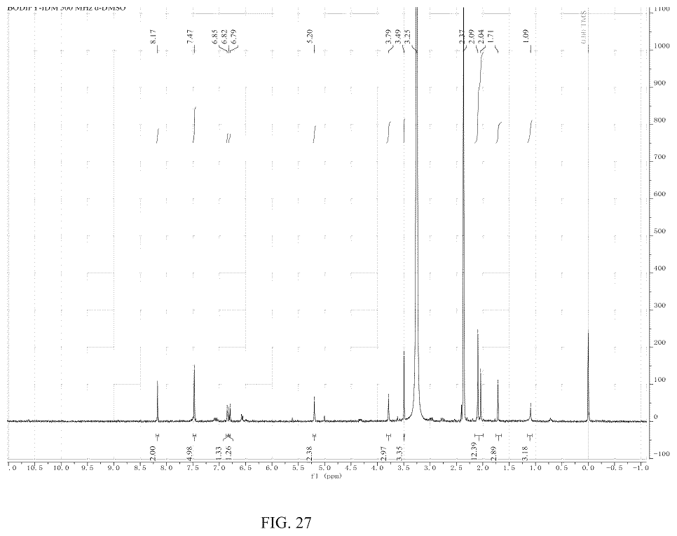

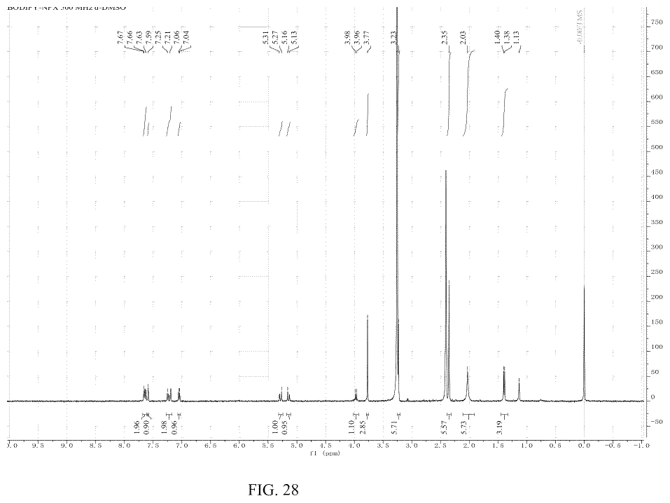

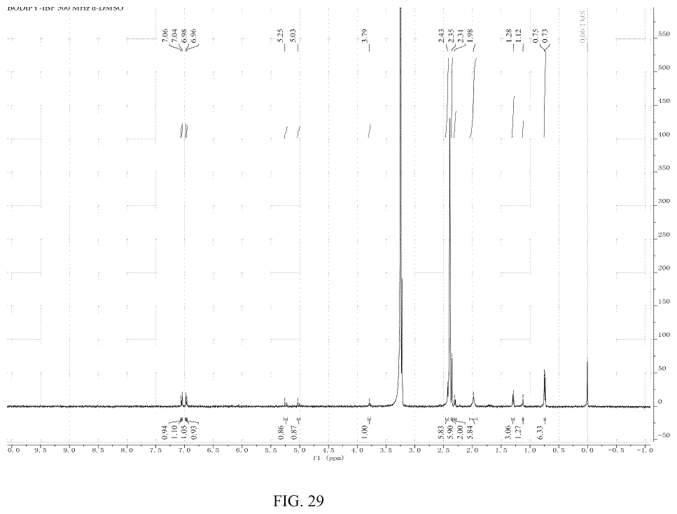

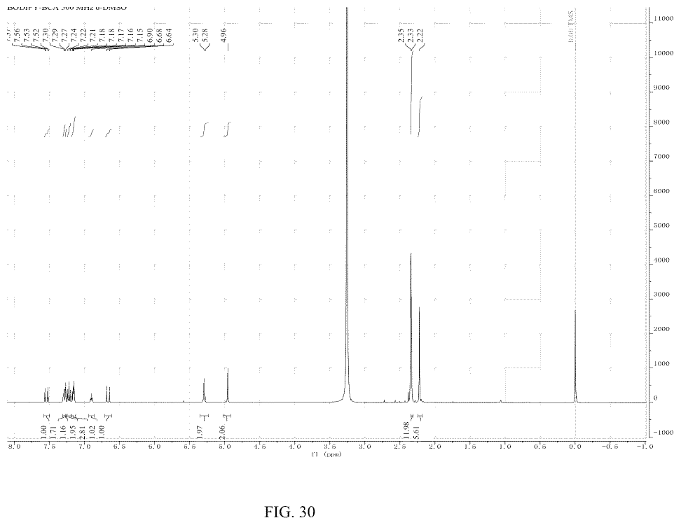

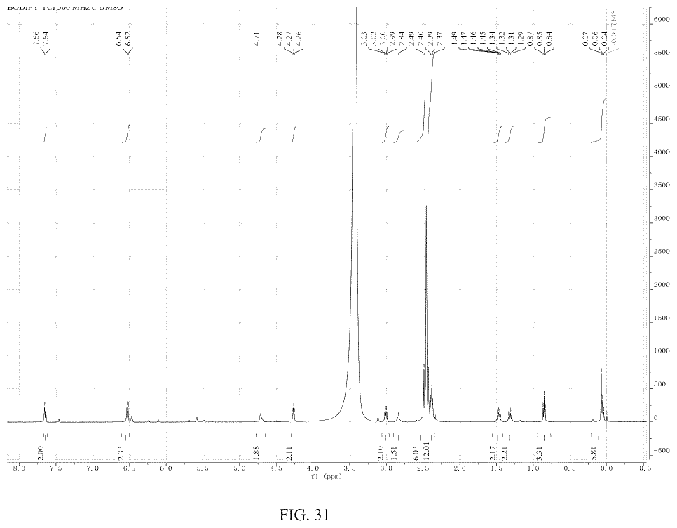

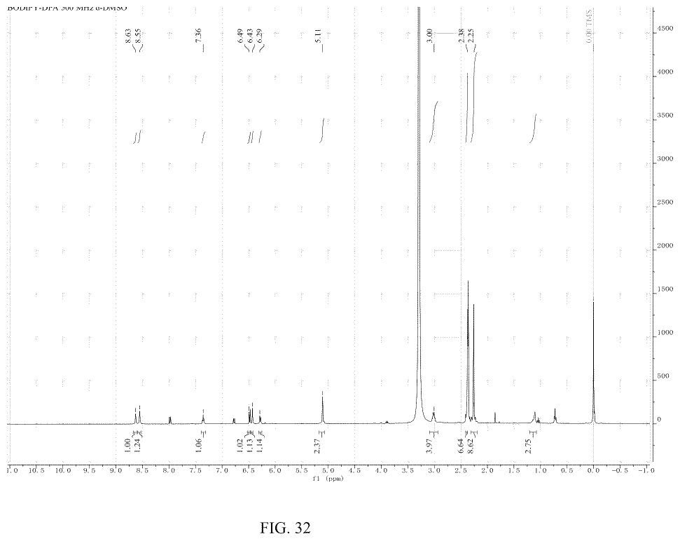

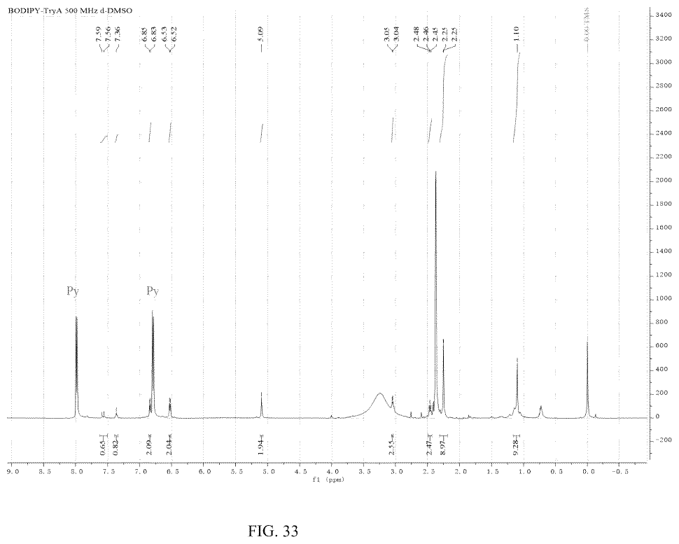

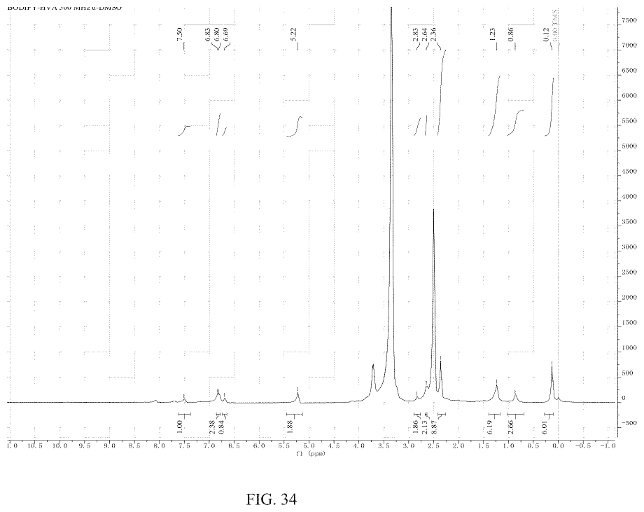

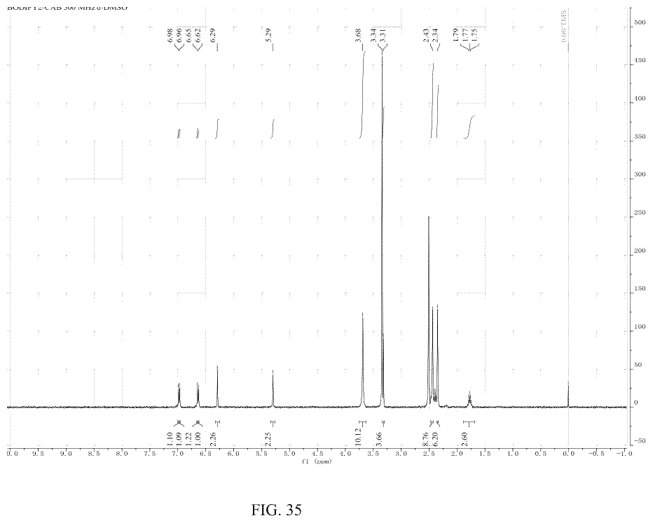

Encouraged by the anti-proliferation effect and tumor retention capability, Os/BC NPs were considered as an applicable agent for light-triggered drug release and photoactivated cancer chemotherapy. The HeLa tumor-bearing mice were randomly divided into six groups (n=5) when the tumors reached at around 100 cm³. PBS, free Cb (8 mg/kg), Os NPs (at the equivalent concentration of Os in Os/BC NPs), BC NPs (at the equivalent concentration of Cb) and Os/BC NPs (at the equivalent concentration of Cb) were intravenously injected on Day 1 (FIG. 6C). At 24 h post injection, NIR-light irradiation (690 nm, 300 mW/cm², 10 min) was applied topically onto the tumor area (FIG. 6D). The formulation injection and light irradiation were repeated on Day 6 and 7, respectively. For evaluating therapeutic efficacy, tumor volume was recorded within the treatment period. Obviously, Group 6 (Os/BC NPs+hv) exhibited the most obvious suppression effect on tumor growth as compared to other groups (FIG. 6E). Group 3 (Os NPs+hv) displayed slightly suppression effect on tumor volume. However, no statistical difference was found between Group 3 and Groups 1, 2, 4 and 5. This result is consistent with the above finding of low cytotoxicity of Os NPs upon light irradiation, which can be explained by the limited phototoxicity of Os(bptpy)₂²⁺. Systemically administration of free chlorambucil (Group 2) did not exhibit detectable anti-tumor effect, which is due to its short circulation time and rapid hydrolysis is in blood.³⁴ On Day 13, we euthanized the mice and excised tumors and organs for ex vivo characterization (FIG. 6F). Tumor weight of Group 6 was significantly lower than other groups, demonstrating the excellent anti-tumor efficacy of Os/BC NPs with NIR light (FIG. 21). Hematoxylin and eosin (H&E) staining assay was conducted to investigate the pathology of the tumors and organs. Obvious necrosis was found in the tumor tissues treated with Os/BC NPs+hv, while negligible cell apoptosis/tissue necrosis were observed in other groups (FIG. 6H). No obvious tissue damage was observed in major organs including heart, lung, liver, spleen, and kidney in all treated mice, indicating negligible in vivo toxicity of the treatments (FIG. 22). Moreover, no significant change of the body weight was observed, further indicating low systemic side effects of our system (FIG. 6G).

### 6.6 Discussion

In summary, we have developed an upconversion-like prodrug photolysis process based on the one-step energy transfer from STPS to BODIPY-based prodrugs, overcoming the limitation of photon energy which must be higher than the S₁ state of photosensitizer. This process depends on the direct activation of the STPS, followed by energy transfer from the T₁ state of STPS to that of prodrug. Such strategy allows the utilization of low-energy photons, such as the NIR photons. Besides of the prolonged excitation wavelength, this strategy demonstrated many other strengths, such as low light irradiance and high photolytic yield, which are presumably explained by reduced energy loss and less photodamage during the photolysis reaction. Notably, such upconversion-like photolysis strategy can be highly modular. A series of prodrugs were fabricated for NIR light-triggered optochemical control of a board range of bioactive molecules, including anti-cancer drugs, anti-inflammation drugs, anaesthesia agents, and biogenic amines. Moreover, PLA-mPEG micellar nanoparticles can enclose the photosensitizer and prodrug, which allowed NIR light-triggered drug release for effective cancer therapy. In all, we have verified a new mechanism for NIR light-triggered upconversion-like photolysis via one-step energy transfer and a nanoparticle-based method for biomedical applications. This study provides new insights for developing photoactivable systems for the application of photopharmacology and photoresponsive drug delivery.

### 6.7. Materials and Methods

6.7.1.

p-nitrophenyl chloroformate were obtained from Sigma-Aldrich (Steinheim, Germany). Osmium (III) chloride hydrate, 4′-(4-bromophenyl)-2,2′:6′,2″-terpyridine, acetoxy-acetyl chloride, ammonium hexafluorophosphate, boron trifluoride etherate, 2,4-dimethylpyrrole pyridine, triethylamine, hydrochloric acid, N, N-diisopropylethylamine, 3-(4,5-dimethyl-2-thiazolyl)-2,5-diphenyl-2-H-tetrazolium bromide (MTT), and all the other chemicals were obtained from Dieckman (Shenzhen, China). Chlorambucil, dimethyl-xanthone acetic acid, naproxen, ibuprofen, indomethacin, 4-benzyloxycinnamic acid, tetracaine, dopamine, tyramine and homoveratrylamine were purchased from Bide Pharm (Shanghai, China). 2′,7′-Dichlorofluorescein diace (DCFH-DA) and 4′,6-diamidino-2-phenylindole (DAPI) were obtained from Thermo Fisher (Heysham, Lancashire, UK). Solvents, including dichloromethane, N, N-Dimethylformamide, dimethyl sulfoxide, acetonitrile, methanol, hexene, ethyl acetate, tetrahydrofuran, were obtained from Oriental (Hong Kong, China). Silica gel columns were purchased from Teledyne ISCO (Lincoln, USA). PLA₅ₖ-mPEG₅ₖ was supplied by Ponsure Biological Co., Ltd. (Shanghai, China).

### 6.7.2 Characterizations

CombiFlash Rf chromatography instrument was purchased from Teledyne ISCO (Lincoln, USA) for chemical purification. Light sources used in this study were purchased from Yuanming Laser (Ningbo, China) and Mightex (CA, USA). The purity verification and photolysis study were conducted with a high-performance liquid chromatography (HPLC) system (Agilent technologies, 1260), attached with C-18 columns which were obtained from Agilent (Santa Clara, California, United States). Nuclear magnetic resonance (NMR) spectroscopy was collected by Bruker AVANCE III HD 500 spectrometer (MA, US). Fluorescence, phosphorescence and UV-vis spectra were collected by a SpectraMax M4 microplate reader (Molecular Devices, CA, US). The particle size and surface charge were recorded by a dynamic light scattering (DLS) device, Zetasizer Nano-ZS90 (Malvern instruments, UK). Transmission electron microscope (TEM) imaging was conducted on a Hitachi HT7700 TEM (Tokyo, Japan). ACEA NovoCyte Quanteon flow cytometer (ACEA Biosciences, CA, USA) was used for flow cytometry. In vivo and ex vivo imaging of the mice were conducted with an In Vivo Imaging System (PerkinElmer, USA). Devices for animal study like including balance and vernier caliper were kindly provided by The Centre for Comparative Medicine Research (CCMR), The University of Hong Kong.

### 6.7.3 Fabrication and Characterization of Nanoparticles

Flash nanoprecipitation method was used to fabricate the photosensitizer/prodrug-loading nanoparticles. The stock solutions of PLA₅ₖ-mPEG₅ₖ (200 mg/mL in acetone), BODIPY-Cb (3×10⁻² M, 24.2 mg/mL in DMSO) and Os(bptpy)₂·2PF₆ (10⁻³ M, 1.26 mg/mL in DMSO) were prepared. Different formulations (Os NPs, BC NPs and Os/BC NPs) were then prepared with optimized ratios of components. Briefly, 224 μL of the mixed stock solution was added into 1.8 mL N₂-saturated water under vortexing. The resulted solutions were added to dialysis bags (M_{W}: 3400 Da) and dialyzed against 4 L of water for 24 h. The water out of dialysis bag was renewed every 8 h. The resulted solution of NPs was collected and filtrated with 220 nm filter. Finally, the NPs solutions were concentrated by ultrafiltration (Mw=100 kDa, 2000×g, 10 min) and stored at 4° C. until use. The concentrations of each component were measured by using HPLC. Loading capacity and encapsulation efficiency were calculated as follow:

$$\begin{matrix}Loading capacity ( \% ) = \frac{weight of loaded payload}{weight of nanoparticles} \times 100 \% & ( 4 ) \\ Encapsulation efficiency ( \% ) = \frac{weight of loaded payload}{weight of fed payload} \times 100 \% & ( 5 )\end{matrix}$$

### 6.7.4 Measurement of Prodrug Photolysis in Nanoparticles

Aqueous solution of Os NPs, BC NPs and Os/BC NPs were diluted to 10⁻³ M (on basis of BODIPY-Cb) and added to 1.5 mL tubes. 690 nm light (100 mW/cm²) was applied topically onto the solution at room temperature for 0-30 min. At each time point, 100 μL sample was collected and dispersed with equal volume (100 μL) of acetonitrile. Then HPLC was used to analyze the prodrug consumption and drug release yield.

### 6.7.5 Cell Culture

Human cervical cancer cells (HeLa) were purchased from Stem Cell Bank, Chinese Academy of Sciences. Cells were cultured in DMEM (Gibco) supplemented with 10% FBS (Gibco) and 100 units/mL antibiotics (Penicillin-Streptomycin, Gibco) at 37° C. in a 5% CO₂ humidified atmosphere.

### 6.7.6 Cytotoxicity Analysis

Cell viabilities were determined by MTT assay. Hela cells were cultured on 96-well plates at a primary density of 5000 cells/well in 100 μL complete DMEM medium and incubated for 24 h. The medium was replaced with the formulations-contained medium (Os NPs, BC NPs and Os/BC NPs) at different concentrations (0-50 μM on basis of BODIPY-Cb). After 4-h incubation, the cells were irradiated by NIR light (690 nm, 100 mW/cm²) for 30 min. MTT solution (10 μL/well) was added after 24 h of incubation. After 3 h, the medium was discarded, and DMSO (100 μL) was added into each well. OD490 values were recorded by plate reader for the calculation of cell viability.

### 6.7.7 Live/Dead Cell Staining

HeLa cells were seeded in confocal dishes at a density of 10000 cells/well and treated with PBS, Os NPs, BC NPs, Os/BC NPs with an equivalent concentration of BODIPY-Cb at 10 μM. For light irradiation, cells were irradiated after 4 h by 690 nm light (100 mW/cm²) for 30 min, followed by 24 h incubation. After washing by PBS and replacing the medium, Calcein-AM and PI were added and the cells were observed by confocal laser scanning microscopy (λex=488 nm, 560 nm).

### 6.7.8 Cell Apoptosis Analysis

HeLa cells were seeded in 6-well plates at a density of 50000 cells/well and treated with free Cb or IR783/BC NPs PBS, Os NPs, BC NPs, Os/BC NPs with an equivalent concentration of BODIPY-Cb at 10 μM for 4 h. Then the cells in the irradiation group were irradiated by light (690 nm, 100 mW/cm²) for 30 min. After 24-h incubation, cells were washed with PBS for 3 times, collected by trypsin digestion, and stained with Annexin-V/FITC apoptosis kit. The cell suspension solutions were analyzed by flow cytometer (λex=488 nm, 560 nm).

### 6.7.9 Animals

BALB/c nude mice (female, 4 weeks, 15-20 g) were obtained from the Experimental Animal Center of University with access to food and water ad libitum. The animal experiment and procedures were approved by the Committee on the Use of Live Animals in Teaching & Research (CULATR), The University of Hong Kong. (Protocol No. 4381-17).

HeLa tumor-bearing mice was obtained by subcutaneously injecting HeLa cells for tumor implantation. HeLa cancer cells were collected by cell culture and trypsin digestion. The cells were washed by PBS and resuspended in DMEM medium without serum, in which collagen and Matrigel were added. 2×10⁶ cells were dispersed in 100 μL mixture, which was subcutaneously injected into the mice at underarm area. After 7 days incubation, the mice with tumor were randomly divided into groups for in vivo experiments.

### 6.7.10 In Vivo Biodistribution

The biodistribution of free drug or nanoparticles in the HeLa tumor-bearing mice was measured by an in vivo fluorescence imaging system. DiR (1,1-dioctadecyl-3,3,3,3-tetramethylindotricarbocyanine iodide) was used the fluorescent dye for labelling the PLA-mPEG NPs during living imaging. 6 mice with HeLa tumors (volumes at about 200 mm³) was randomly divided into two groups and treated with free DiR or DiR in PLA-mPEG NPs via intravenous injection with a dose of DiR at 100 μg/kg. The in vivo fluorescence imaging was performed at 1, 2, 8 and 24 h post injection and anesthesia at each time point. At 24 h, all the mice were euthanized. Tumors and major organs (heart, liver, spleen, lung, kidney) were excised for ex vivo imaging (λex=710 nm, λem=780 nm).

### 6.7.11 In Vivo Light-Triggered Prodrug Activation and Tumor Inhibition

The anti-tumor efficacy of the formulations in presence or absence of light was investigated with HeLa tumor-bearing mice. 7 days after tumor implantation, the mice with tumors at the volumes at around 100 mm³ were randomly divided into 6 groups (5 mice per group). Different treatments were administrated to each group: (1) PBS; (2) free chlorambucil; (3) Os NPs plus light irradiation; (4) BC NPs plus light irradiation; (5) Os/BC NPs; (6) Os/BC NPs plus light irradiation. The formulations were injected intravenously on Day 1 and Day 6, of which the dose was set as 8 mg/kg. For the groups with light irradiation, light irradiation (690 nm, 300 mW/cm², 10 min) were performed 24 h post injection (on Day 2 and Day 7). Tumor sizes and body weights were measured during the period, and the tumor volume was calculated as V=½×width²×length. On Day 13, all of the mice were euthanized, and the tumors and major organs were excised and sliced for H&E staining and histochemical analysis. All the histological study were kindly performed in blinded fashion by a pathologist from the Department of Pathology, The University of Hong Kong.

### 6.7.12 Statistical Analysis

All experiments were conducted three times or more independently (n≥3). Data were presented as the mean standard deviation (SD). The one-way ANOVA-LSD and Independent-Samples t-test were adopted to determine the statistical significance of differences by Graphpad Prism 8.0 software (***p<0.001, **p<0.01, and *p<0.05).

### 6.7.13 Synthesis

6.7.13.1

The synthesis steps of two BODIPY photocages, compound 1 and 2 (Scheme S1), have been reported in our previous studies.

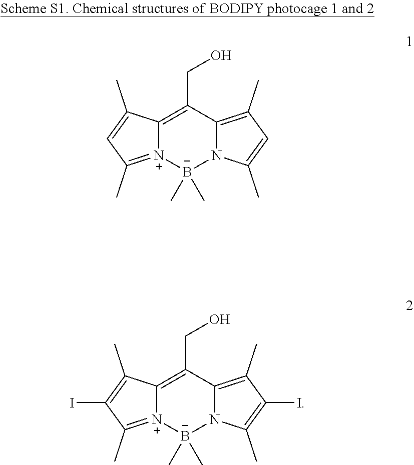 [SMILES: CC1=CC(C)=[N+]2C1=C(CO)c1c(C)cc(C)n1[B-]2(C)C ; CC1=C(I)C(C)=[N+]2C1=C(CO)c1c(C)c(I)c(C)n1[B-]2(C)C]

6.7.13.2 Synthesis of [Os(bptpy)₂]·2PF₆ (Compound 3)

The synthesis of [Os(bptpy)₂]·2PF₆ was conducted based on the reported method with modifications.

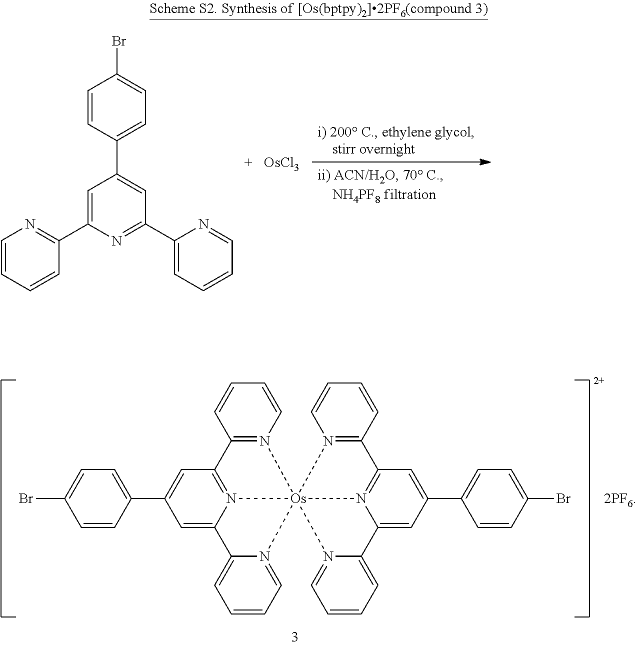

Osmium (III) chloride hydrate (314.6 mg, 1 mmol) and 4′-(4-bromophenyl)-2,2′:6′,2″-terpyridine (766.5 mg, 2 mmol) were dissolved in 50 mL ethylene glycol and protected with nitrogen. The solution was heated to 200° C. and refluxed overnight under continuous stirring. The solution changed to dark purple after the reaction. The solution was dropwise added into excess water (500 mL) under vigorously stirring. The resulted purple precipitates were collected by filtration, washed by water and THF and dried. Then the resulted powder was dissolved in ACN (150 mL) and heated at 70° C. Ammonium hexafluorophosphate (16.3 g, 10 mmol) was dissolved in water and added under stirring. After 2 h, the solution was cooled down, and the product was collected by filtration as dark purple precipitate and washed by water and ACN without further purification. ¹H NMR (500 MHz, DMSO-d6): δ (ppm), 9.52 (s, 4H), 9.09-9.10 (d, 4H), 8.38-8.40 (d, 4H), 7.99-8.01 (d, 4H), 7.92-7.95 (t, 4H), 7.43-7.44 (d, 4H), 7.20-7.23 (t, 4H). m/z=968.03, found 968.00.

### 6.7.13.3 General Procedures for the Preparation of BODIPY Ester Prodrugs (4-9, 14)

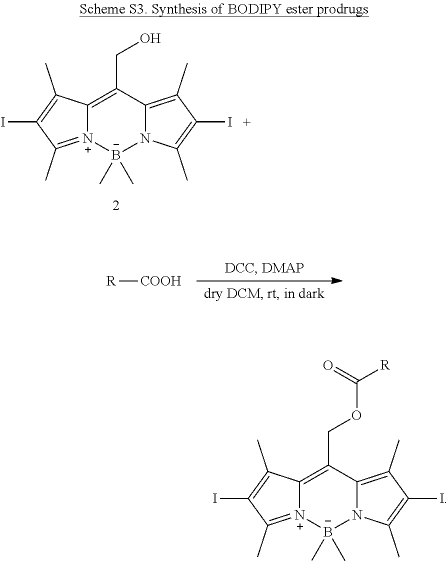

7.5 mg BODIPY photocage 2 (0.014 mmol) was dissolved in 300 μL dry dichloromethane and protected in dark environment. Drug with carboxyl group (0.021 mmol), N, N′-dicyclohexylcarbodiimide (DCC) (6.9 mg, 0.0334 mmol), 4-dimethylaminopyridine (DMAP) (0.17 mg, 0.0014 mmol) were added to a sealable flask under the protection of nitrogen. Added 500 μL dry dichloromethane into the flask and stirred for 30 min, then injected the solution of 2 dropwise. The flask was placed in dark environment with tin foil covered on the surface. After 12 h, followed by the thin-layer chromatography to verify the complete reaction, the solvent was removed by evaporation. The residue was subjected to column chromatography on silica gel for purification.

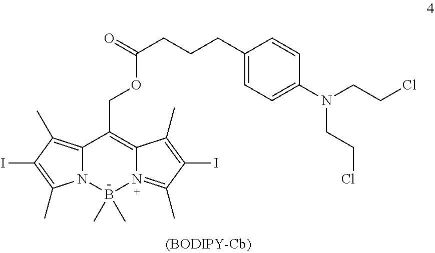

4: Purple-red solid. ¹H NMR (500 MHz, DMSO-d6): δ 6.96-6.98 (m, 2H), 6.62-6.64 (m, 2H) (aromatic CH), 5.34 (s, 2H), 3.69 (m, 8H) (CH₂), 3.31 (s, 6H), 2.36-2.45 (m, 9H), 1.76-1.79 (t, 3H), 0.14 (s, 6H) (CH₃). [M+H]⁺: 809.17, found 809.20. 89% yield.

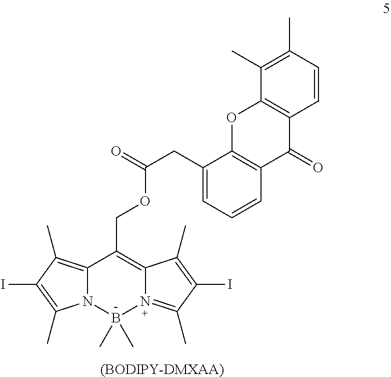

5: Dark-red solid. ¹H NMR (500 MHz, DMSO-d6): δ 8.19 (m, 2H), 7.87 (s, 1H), 7.68-7.70 (d, 1H), 7.48-7.50 (d, 1H) (aromatic CH), 4.71 (s, 2H), 3.00 (s, 2H) (CH₂), 2.34-2.43 (m, 9H), 1.20-1.21 (d, 9H), 0.16 (s, 6H) (CH₃). 92% yield.

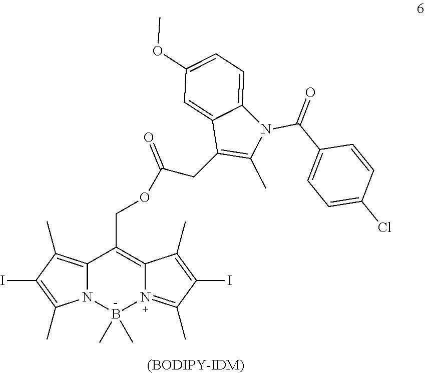

6: Purple-red solid. ¹H NMR (500 MHz, DMSO-d6): δ 8.17 (s, 2H), 7.47 (m, 5H) (aromatic CH), 6.79-6.85 (m, 2H), 5.20 (s, 2H) (CH₂), 3.79 (s, 3H), 3.49 (s, 3H), 2.04-2.09 (m, 12H), 1.71 (s, 3H), 1.09 (s, 3H) (CH₃). [M+H]⁺: 862.75, found 862.70. 75% yield.

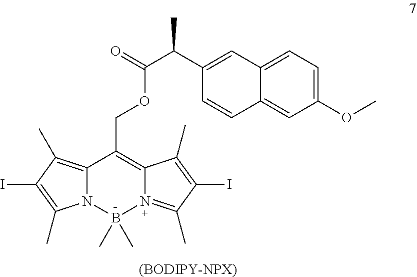

7: Purple-red solid. ¹H NMR (500 MHz, DMSO-d6): δ 7.63-7.67 (m, 2H), 7.59 (s, 1H), 7.21-7.25 (m, 2H), 7.04-7.06 (d, 1H) (aromatic CH), 5.27-5.31 (d, 1H), 5.13-5.16 (d, 1H) (CH₂), 3.96-3.98 (m, 1H) (CH), 3.77 (s, 3H), 3.23 (m, 6H), 2.35 (s, 6H), 2.03 (s, 6H), 1.38-1.40 (d, 3H) (CH₃). [M+H]⁺: 735.22, found 735.20. 88% yield.

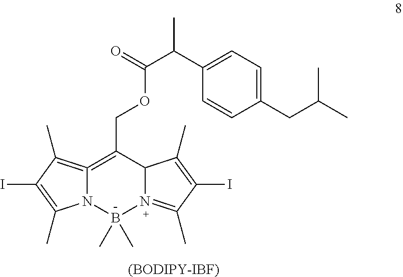

8: Red solid. ¹H NMR (500 MHz, DMSO-d6): δ 7.04-7.06 (D, 2H), 6.96-6.98 (d, 2H) (aromatic CH), 5.25 (d, 1H), 5.03 (d, 1H), 3.79 (d, 1H), 2.43 (m, 6H), 2.35 (m, 6H), 2.29-2.31 (d, 2H), 1.98 (m, 6H), 1.28-1.30 (d, 3H), 1.12 (s, 1H), 0.73-0.75 (d, 6H) (CH₃). [M+H]⁺: 711.25, found 711.20. 88% yield.

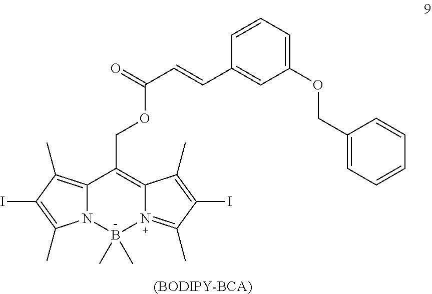

9: Purple-red solid. ¹H NMR (500 MHz, DMSO-d6): δ 7.52-7.57 (d, 2H), 7.15-7.30 (m, 8H) (aromatic CH), 6.90 (m, 1H), 6.64-6.68 (d, 1H), 5.28-5.30 (s, 2H), 4.96 (s, 2H), 2.33-2.35 (m, 12H), 2.22 (s, 6H) (CH₃). 65% yield.

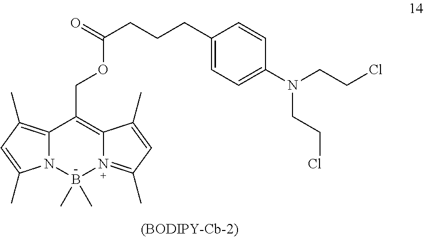

14: Orange solid. ¹H NMR (500 MHz, DMSO-d6): δ 6.96-6.98 (d, 2H), 6.62-6.65 (d, 2H), 6.29 (s, 2H) (aromatic CH), 5.29 (s, 2H), 3.68 (s, 10H), 3.31 (m, 4H) (CH₂), 2.43 (m, 9H), 2.34 (m, 6H), 1.75-1.79 (m, 3H) (CH₃). [M+H]⁺: 557.38, found 557.40. 78% yield.

### 6.7.13.4 General Procedures for the Preparation of BODIPY Carbamate Prodrugs (10-13)

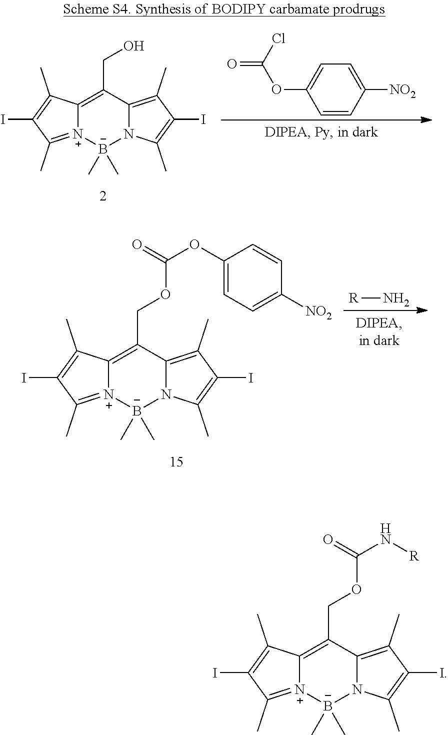

BODIPY photocage 2 (50.0 mg, 0.096 mmol) was added to a 50 mL sealable flask. The flask was then vacuumed by pump and refilled by nitrogen gas. Dry dichloromethane (8 mL) was added to dissolve the solid under stirring. The flask was cooled to 0° C. in an ice-water bath. N, N-diisopropylethylamine (0.168 mL, 0.96 mmol) was added with a syringe with needle. Then p-nitrophenyl chloroformate (193.1 mg, 0.96 mmol) was dissolved in dry dichloromethane (2 mL) and added into the above solution dropwise. Pyridine (31 μL, 0.38 mmol) was added with a syringe with needle. The flask was placed in dark environment with tin foil covered on the surface. After 12 h, followed by the thin-layer chromatography to verify the complete reaction, the solvent was removed by evaporation. The product (15, BODIPY-p-nitrobenzene) was purified by chromatography on a silica column, flowed by vacuum-drying for at least 24 h. Yield: 58%.

BODIPY-p-nitrobenzene (15, 20 mg, 0.029 mmol) was dissolved with tetrahydrofuran (2 mL) in a 25 mL sealable flask, which was vacuumed by pump and refilled by nitrogen gas. Under stirring at room temperature, the drug with amino group (0.087 mmol) was dissolved in 0.5 mL DMF and added to the solution with a syringe with needle. N, N-diisopropylethylamine (18 μL) was then added to the mixture. The flask was placed in dark environment with tin foil covered on the surface. After 4 h, the solvent was removed by evaporation and the product was collected by high-performance liquid chromatography.

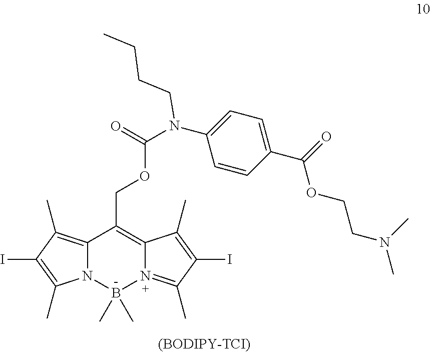

10: Dark-red solid. ¹H NMR (500 MHz, DMSO-d6): δ 7.64-7.66 (d, 2H), 6.52-6.54 (d, 2H) (aromatic CH), 4.71 (m, 2H), 4.26-4.28 (t, 2H), 2.99-3.03 (m, 2H), 2.84 (s, 2H) (CH₂), 2.49 (m, 6H), 2.37-2.40 (m, 12H) (CH₃), 1.45-1.79 (m, 2H), 1.29-1.34 (m, 2H) (CH₂), 0.84-0.87 (t, 3H), 0.04-0.07 (m, 6H) (CH₃). 26% yield.

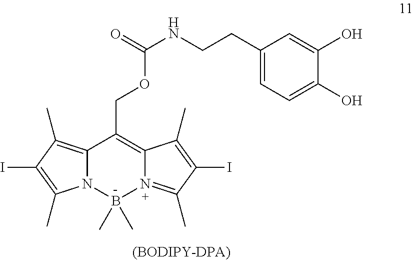

11: Dark-red solid. ¹H NMR (500 MHz, DMSO-d6): δ 8.63 (s, 1H), 8.55 (s, 1H) (OH), 7.36 (t, 1H) (NH), 6.49 (d, 1H), 6.43 (d, 1H), 6.29 (m, 1H) (aromatic CH), 5.11 (s, 2H), 3.97 (m, 4H) (CH₂), 2.38 (m, 6H), 2.25 (s, 9H), 1.10-1.14 (m, 3H) (CH₃). 43% yield.

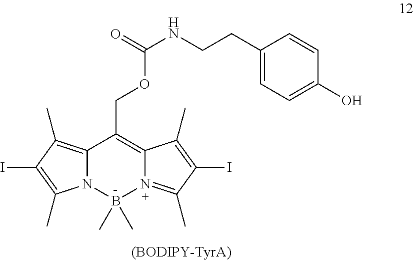

12: Dark-red solid. ¹H NMR (500 MHz, DMSO-d6): δ 7.56-7.59 (m, 1H) (OH), 6.83-6.85 (d, 2H), 6.52-6.53 (d, 2H) (aromatic CH), 6.49 (d, 1H), 6.43 (d, 1H), 6.29 (m, 1H) (aromatic CH), 5.09 (s, 2H), 3.04-3.05 (m, 2H), 2.45-2.48 (m, 2H) (CH₂), 2.25 (s, 9H), 1.10 (s, 9H) (CH₃). 37% yield.

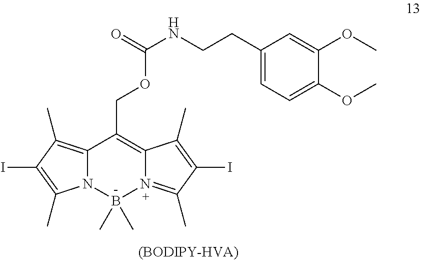

13: Dark-red solid. ¹H NMR (500 MHz, DMSO-d6): δ 7.50 (m, 1H) (NH), 6.80-6.83 (m, 2H), 6.69 (s, 1H) (aromatic CH), 5.22 (s, 2H), 2.84 (m, 2H), 2.63 (m, 2H) (CH₂), 2.36 (s, 9H), 1.23 (m, 6H), 0.66 (s, 3H) (aromatic CH), 0.12 (s, 6H). 50% yield.
6.7.13.5 Measurement of Energy Transfer Quenching Rate Constants (k_{TTET})

The energy transfer quenching rate constants (k_{TTET}) was measured by Stern-Volmer experiment. As the representative of BODIPY prodrugs, BODIPY-Cb (compound 4) was used as the energy receptor while Os(bptpy)₂²⁺ was the energy donor during the quenching process. 10 μM of Os(bptpy)₂²⁺ solutions were prepared in the present of different concentrations of BODIPY-Cb (0, 1, 2, 3, 5, 7.5 μM), of which the solvent was mixed by 88% methanol, 2% acetone and 10% dichloromethane. Oxygen was removed by nitrogen bubbling (50 mL/min) for 10 min.

Quenching constants (k_{q}) of Os(bptpy)₂²⁺ in present of BODIPY-Cb were calculated by the followed equation (1):

$$\frac{{I}_{0}}{I} = 1 + {k}_{q} [ Q ] \qquad ( 1 )$$

(I₀: phosphorescence intensity of the Os(bptpy)₂²⁺ solution; I: phosphorescence intensity of Os(bptpy)₂²⁺ solution in presence of BODIPY-Cb; [Q]: concentration of BODIPY-Cb.)

Energy transfer quenching rate constants of this TTET process, quantified as k_{TTET}, can be calculated based on the below equation (2)

$${k}_{TTET} = \frac{{k}_{q}}{{τ}_{0}} \qquad ( 2 )$$

(τ₀: phosphorescence lifetime of Os(bptpy)₂²⁺ in N₂-saturated solution without quencher.)

For Os(bptpy)₂²⁺, τ₀=0.20 μs. The k_{q} of Os(bptpy)₂²⁺ in present of BODIPY-Cb was determined to be (5.491±0.282)×10⁴ M⁻¹. Thus, the energy transfer quenching rate constant of the TTET between Os(bptpy)₂²⁺ and BODIPY-Cb was calculated as:

$$( 2.718 \pm 0.14 ) \times {10}^{11} {M}^{- 1} {s}^{- 1} .$$

### 6.7.13.6 Quantitative Analysis of BODIPY Prodrug Photolysis

The photolysis efficiency of BODIPY prodrugs were compared in different conditions, including different excitation wavelengths (690 nm or 530 nm), different ratios of Os(bptpy)₂²⁺, and different oxygen contents. A 100 μL mixture of BODIPY-Cb (compound 4) (100 μM) and Os(bptpy)₂²⁺ (0 eq (0 μM), 0.05 eq (5 μM), 0.1 (10 μM)) was saturated with nitrogen or not and irradiated by 690 nm light (100 mW/cm², 5 min) or 530 nm light. The solvent was 88% methanol mixed by 10% dichloromethane and 2% acetone. For the N₂-saturated groups, after dissolving the molecules, the solution was degassed with a vacuum pump for 10 min followed by gentle N₂ blowing. After light irradiation, the resulted mixtures were refilled to 100 μL with methanol to avoid the error caused by solvent evaporation. The resulted solution was loaded onto the sampler and analyzed by high-performance liquid chromatography. An elution method was used to separate free drug, BODIPY photocage and prodrug on the C18 column.

### 6.7.13.7 Measurement of the Photoreaction Quantum Yields

Quantum yields of the photoreactions (Φ) are defined as:

$$Φ = \frac{number of reacted molecule per time unit}{number of absorbed photons per time unit} \qquad ( 3 )$$

The number of reacted molecules (e.g., consumption of prodrugs or generation of free drugs) were determined by HPLC. The absorbed photon number was determined by Reinecke's salt actinometry at excitation wavelengths of 530 nm and 690 nm^{35, 36}. The power densities were 50 mW/cm² (530 nm) and 100 mW/cm² (690 nm).

### Exemplary Products, Systems and Methods are Set Out in the Following Items:

1. A composition, comprising:
   a photosensitizer capable of singlet-to-triplet (S-T) activation at a near-infrared (NIR) wavelength; a cleavable moiety to accept triplet-triplet energy transfer from the photosensitizer in a higher energy state to cause cleavage of the cleavable moiety; and a releasable moiety releasable from the composition upon cleavage of the cleavable moiety,
   wherein the composition does not comprise an annihilator.
2. The composition of item 1, wherein the single-to-triplet activation is from a singlet (S₀) state to a triplet (T₁) state.
3. The composition of item 2, wherein the single-to-triplet activation is directly from a singlet (S₀) state to a triplet (T₁) state.
4. The composition of item 2, wherein the triplet-triplet energy transfer is from the triplet (T₁) state of the photosensitizer to the cleavable moiety.
5. The composition of any one of items 2-4, wherein the triplet (T₁) state of the photosensitizer is higher than the triplet state of the cleavable moiety.
6. The composition of any one of items 1-5, wherein the photosensitizer comprises a transition metal-porphyrin.
7. The composition of item 6, wherein the transition metal is osmium (Os).
8. The composition of any one of items 1-6, wherein the photosensitizer is Os (II) bromophenyl terpyridine complex (Os(bptpy)₂²⁺).
9. The composition of any one of items 1-8, wherein the near infrared (NIR) wavelength is between about 650 nm and about 750 nm.
10. The composition of item 9, wherein the near infrared (NIR) wavelength is about 690 nm.
11. The composition of item 1, wherein the photosensitizer has an excitation wavelength of between about 650 nm and about 750 nm.
12. The composition of item 11, wherein the photosensitizer has an excitation wavelength of about 690 nm.
13. The composition of any one of items 1-12, wherein the cleavable moiety is photocleavable.
14. The composition of any one of items 1-13, wherein the cleavable moiety is BODIPY.
15. The composition of any one of items 1-14, wherein the releasable moiety comprises a drug.
16. The composition of item 15, wherein the drug is an anti-inflammatory drug, an anti-cancer drug or an anti-angiogenesis drug.
17. The composition of item 15 or 16, wherein the drug is selected from the group consisting of chlorambucil (CAB), vadimezan, indomethacin, naproxen, ibuprofen, benzyloxycinnamic acid, tetracaine, dopamine, tyramine and homoveratrylamine.
18. The composition of any one of items 1-17, wherein the composition comprises a prodrug, the prodrug comprising the cleavable moiety and the releasable moiety.
19. The composition of item 18, wherein the prodrug comprises a drug that is linked to BODIPY.
20. The composition of claim 18, wherein the cleavable moiety is BODIPY, and wherein the releasable moiety comprises a drug selected from the group consisting of chlorambucil, vadimezan, indomethacin, naproxen, ibuprofen, benzyloxycinnamic acid, tetracaine, dopamine, tyramine and homoveratrylamine.
21. The composition of item 18, wherein the prodrug is represented by the structure:

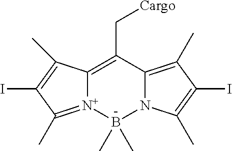

wherein ‘Cargo’ comprises the releasable moiety.
22. The composition of item 21, wherein Cargo comprises a drug.
23. The composition of any one of items 18-22, wherein the prodrug is selected from the group consisting of:

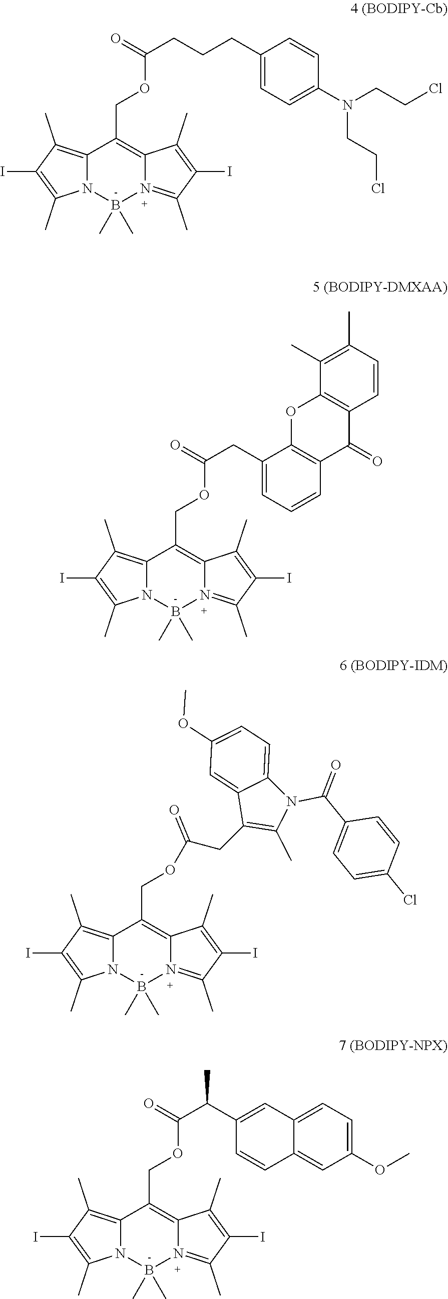

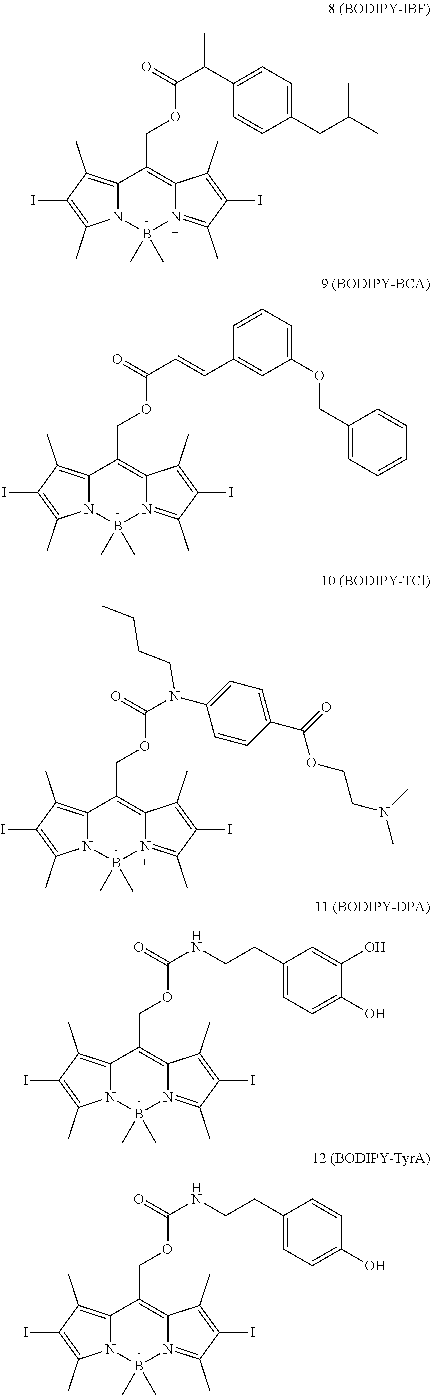

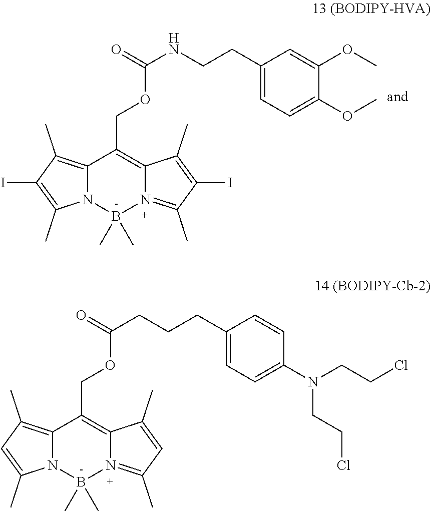

24. The composition of any one of items 1-23, wherein at least about 50% of the cleavable moiety is released.
25. The composition of any one of items 1-24, wherein at least about 80% of the cleavable moiety is released.
26. The composition of any one of items 1-25, wherein at least about 90% of the cleavable moiety is released.
27. The composition of any one of items 1-26, further comprising a carrier material.
28. The composition of item 27, wherein the carrier material comprises the photosensitize, the releasable moiety and the cleavable moiety.
29. The composition of item 27 or 28, wherein the carrier material comprises a polymer.
30. The composition of any one of items 27-29, wherein the carrier material comprises a particle.
31. The composition of item 30, wherein the particle has an average diameter of less than about 1 mm.
32. The composition of any one of items 27-31, wherein the carrier material comprises a film.
33. The composition of any one of items 27-32, wherein the carrier material comprises a polymeric micelle.
34. A composition, comprising:
   a photosensitizer capable of singlet-to-triplet (S-T) activation upon exposure to a near-infrared (NIR) wavelength; and
   an active moiety to accept triplet-triplet energy transfer from the photosensitizer in a higher energy state to cause a chemical reaction within the active moiety;
   wherein the energy transferred from the photosensitizer is sufficient to cause the chemical reaction in the active moiety; and
   wherein the composition does not comprise an annihilator.
35. A method, comprising:
   applying to a subject a composition comprising a photosensitizer capable of singlet-to-triplet (S-T) activation upon exposure to a near-infrared (NIR) wavelength; a cleavable moiety to accept triplet-triplet energy transfer from the photosensitizer in a higher energy state to cause cleavage of the cleavable moiety; and a releasable moiety releasable from the composition upon cleavage of the cleavable moiety; and
   applying near-infrared (NIR) light to the subject to cause the cleavage of the cleavable moiety and release of the releasable moiety,
   wherein the triplet-triplet energy is not transferred via an annihilator.
36. The method of item 35, wherein the light is coherent.
37. The method of item 35, wherein the light is noncoherent.
38. The method of any one of items 35-37, wherein the composition comprises a prodrug, the prodrug comprising the cleavable moiety and the releasable moiety.
39. A prodrug comprising a cleavable moiety and a releasable moiety, wherein the cleavable moiety is BODIPY, and wherein the releasable moiety comprises a drug selected from the group consisting of vadimezan, indomethacin, naproxen, ibuprofen, benzyloxycinnamic acid, tetracaine, dopamine, tyramine and homoveratrylamine.
40. A compound selected from the group consisting of:

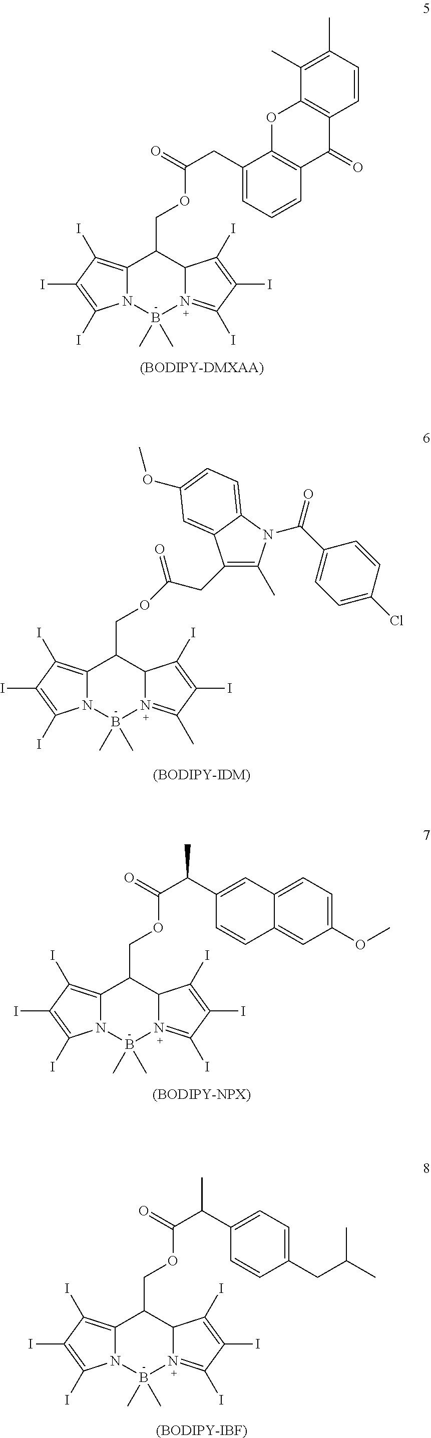

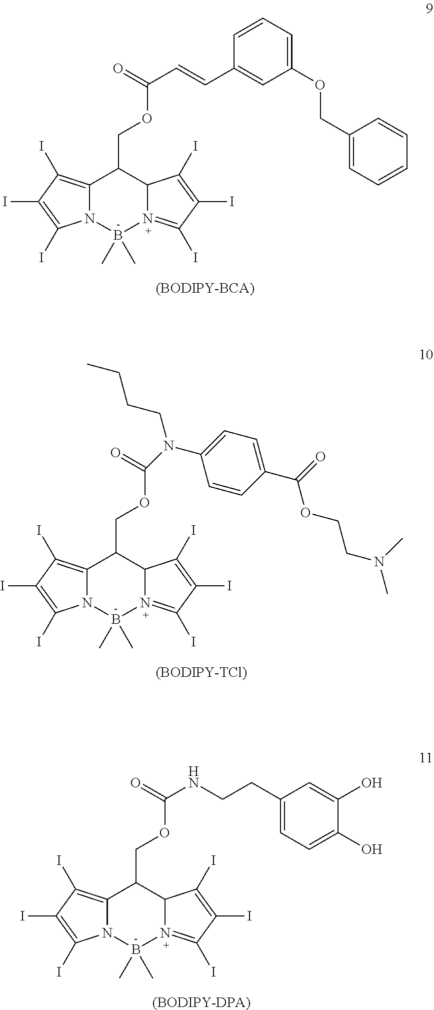

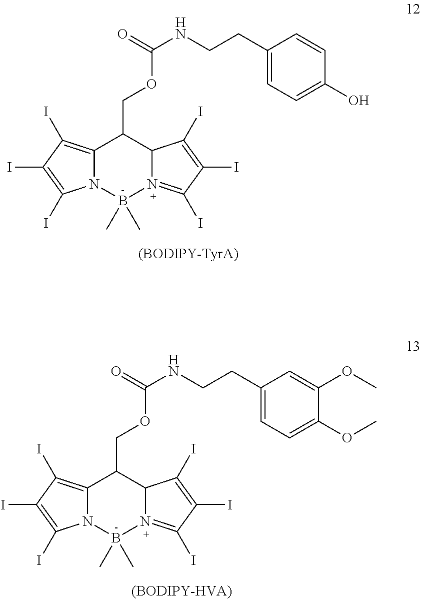

41. The compound of item 40, which is selected from the group consisting of formula 5, 6, 8, and 13.
42. A compound of formula 5.
43. A compound of formula 6.
44. A compound of formula 7.
45. A compound of formula 8.
46. A compound of formula 9.
47. A compound of formula 10.
48. A compound of formula 11.
49. A compound of formula 12.
50. A compound of formula 13.

Those skilled in the art will recognize, or be able to ascertain many equivalents to the specific embodiments of the invention described herein using no more than routine experimentation. Such equivalents are intended to be encompassed by the following claims.

All publications, patents and patent applications mentioned in this specification are incorporated herein by reference in their entireties into the specification to the same extent as if each individual publication, patent or patent application was specifically and individually indicated to be incorporated by reference in its entirety.

Citation or discussion of a reference herein shall not be construed as an admission that such is prior art to the present invention.

### REFERENCES

1. Weinstain, R.; Slanina, T.; Kand, D.; Klan, P., Visible-to-NIR-light activated release: from small molecules to nanomaterials. Chem. Rev. 2020, 120 (24), 13135-13272.
2. Rwei, A. Y.; Wang, W.; Kohane, D. S., Photoresponsive nanoparticles for drug delivery. Nano today 2015, 10 (4), 451-467.
3. Liu, J.; Kang, W.; Wang, W., Photocleavage-based Photoresponsive Drug Delivery. Photochem. Photobiol. 2022, 98 (2), 288-302.
4. Lv, W.; Long, K.; Yang, Y.; Chen, S.; Zhan, C.; Wang, W., A red light-triggered drug release system based on one-photon upconversion-like photolysis. Advanced Healthcare Materials 2020, 9 (21), 2001118.
5. Long, K.; Wang, Y.; Lv, W.; Yang, Y.; Xu, S.; Zhan, C.; Wang, W., Photoresponsive prodrug-dye nanoassembly for in-situ monitorable cancer therapy. Bioengineering & Translational Medicine 2022, 7 (3), e10311.
6. Wong, P. T.; Choi, S. K., Mechanisms of drug release in nanotherapeutic delivery systems. Chem. Rev. 2015, 115 (9), 3388-3432.
7. Lin, M.; Gao, Y.; Hornicek, F.; Xu, F.; Lu, T. J.; Amiji, M.; Duan, Z., Near-infrared light activated delivery platform for cancer therapy. Advances in colloid interface science 2015, 226, 123-137.
8. Wright, C. W.; Guo, Z. F.; Liang, F. S., Light control of cellular processes by using photocaged abscisic acid. ChemBioChem 2015, 16 (2), 254-261.
9. Long, K.; Yang, Y.; Lv, W.; Jiang, K.; Li, Y.; Lo, A. C. Y.; Lam, W. C.; Zhan, C.; Wang, W., Green light-triggered intraocular drug release for intravenous chemotherapy of retinoblastoma. Advanced Science 2021, 8 (20), 2101754.
10. Liu, Y.; Long, K.; Kang, W.; Wang, T.; Wang, W., Optochemical Control of Immune Checkpoint Blockade via Light-Triggered PD-L1 Dimerization. Advanced NanoBiomed Research 2022, 2 (6), 2200017.
11. Wang, T.; Long, K.; Zhou, Y.; Jiang, X.; Liu, J.; Fong, J. H.; Wong, A. S.; Ng, W.-L.; Wang, W., Optochemical Control of mTOR Signaling and mTOR-Dependent Autophagy. ACS Pharmacology Translational Science 2022, 5 (3), 149-155.
12. Singh, P. K.; Majumdar, P.; Singh, S. P., Advances in BODIPY photocleavable protecting groups. Coord. Chem. Rev. 2021, 449, 214193.
13. Shen, J.; Zhao, L.; Han, G., Lanthanide-doped upconverting luminescent nanoparticle platforms for optical imaging-guided drug delivery and therapy. Adv. Drug Del. Rev. 2013, 65 (5), 744-755.
14. Wang, Y.; Zheng, K.; Song, S.; Fan, D.; Zhang, H.; Liu, X., Remote manipulation of upconversion luminescence. Chem. Soc. Rev. 2018, 47 (17), 6473-6485.
15. Zhao, J.; Ji, S.; Guo, H., Triplet-triplet annihilation based upconversion: from triplet sensitizers and triplet acceptors to upconversion quantum yields. RSC Advances 2011, 1 (6), 937-950.
16. Huang, L.; Kakadiaris, E.; Vaneckova, T.; Huang, K.; Vaculovicova, M.; Han, G., Designing next generation of photon upconversion: Recent advances in organic triplet-triplet annihilation upconversion nanoparticles. Biomaterials science 2019, 201, 77-86.
17. Lv, W.; Li, Y.; Li, F.; Lan, X.; Zhang, Y.; Du, L.; Zhao, Q.; Phillips, D. L.; Wang, W., Upconversion-like photolysis of BODIPY-based prodrugs via a one-photon process. J. Am. Chem. Soc. 2019, 141 (44), 17482-17486.
18. Long, K.; Han, H.; Kang, W.; Lv, W.; Wang, L.; Wang, Y.; Ge, L.; Wang, W., One-photon red light-triggered disassembly of small-molecule nanoparticles for drug delivery. Journal of nanobiotechnology 2021, 19 (1), 1-14.
19. Amemori, S.; Sasaki, Y.; Yanai, N.; Kimizuka, N., Near-Infrared-to-Visible Photon Upconversion Sensitized by a Metal Complex with Spin-Forbidden yet Strong S0-T1 Absorption. J. Am. Chem. Soc. 2016, 138 (28), 8702-5.
20. Sasaki, Y.; Amemori, S.; Kouno, H.; Yanai, N.; Kimizuka, N., Near infrared-to-blue photon upconversion by exploiting direct S-T absorption of a molecular sensitizer. Journal of Materials Chemistry C 2017, 5 (21), 5063-5067.
21. Vickerman, B. M.; Zywot, E. M.; Tarrant, T. K.; Lawrence, D. S. J. N. R. C., Taking phototherapeutics from concept to clinical launch. 2021, 5 (11), 816-834.
22. Ankenbruck, N.; Courtney, T.; Naro, Y.; Deiters, A., Optochemical control of biological processes in cells and animals. Angew. Chem. Int. Ed. 2018, 57(11), 2768-2798.
23. Bardhan, A.; Deiters, A., Development of photolabile protecting groups and their application to the optochemical control of cell signaling. Curr. Opin. Struct. Biol. 2019, 57, 164-175.
24. Ly, J. T.; Presley, K. F.; Cooper, T. M.; Baldwin, L. A.; Dalton, M. J.; Grusenmeyer, T. A., Impact of iodine loading and substitution position on intersystem crossing efficiency in a series of ten methylated-meso-phenyl-BODIPY dyes. Physical Chemistry Chemical Physics 2021, 23 (21), 12033-12044.
25. Adams, M. L., Lavasanifar, A. and Kwon, G. S., Amphiphilic block copolymers for drug delivery. Journal of pharmaceutical sciences 2003, 92 (7), 1343-1355.
26. Zhao, W.; Zhao, Y.; Wang, Q.; Liu, T.; Sun, J.; Zhang, R., Remote light-responsive nanocarriers for controlled drug delivery: Advances and perspectives. Small 2019, 15 (45), 1903060.
27. Zhang, A.; Jung, K.; Li, A.; Liu, J.; Boyer, C., Recent advances in stimuli-responsive polymer systems for remotely controlled drug release. Prog. Polym. Sci. 2019, 99, 101164.
28. Yang, Y.; Long, K.; Wang, Y.; Li, L.; Shi, J.; Liu, J.; Kong, L.; Yu, L.; Ding, J.; Huang, Z., NIR light-triggered quantitative pulsed drug release. Advanced Healthcare Materials 2022, 11 (8), 2102362.
29. More, G. S.; Thomas, A. B.; Chitlange, S. S.; Nanda, R. K.; Gajbhiye, R. L., Nitrogen mustards as alkylating agents: a review on chemistry, mechanism of action and current USFDA status of drugs. Anticancer Agents Med. Chem. 2019, 19 (9), 1080-1102.
30. Pedersen, P. J.; Christensen, M. S.; Ruysschaert, T.; Linderoth, L.; Andresen, T. L.; Melander, F.; Mouritsen, O. G.; Madsen, R.; Clausen, M. H., Synthesis and biophysical characterization of chlorambucil anticancer ether lipid prodrugs. J. Med. Chem. 2009, 52 (10), 3408-3415.
31. Zeng, L.; Jiang, L.-H.; Li, J.-Y.; Huang, L.; Chen, Y.; Yu, N.; Wang, L.; Huang, K.; Peng, J.; Han, G., Metal-free Far-red Light-driven-Photolysis via Triplet Fusion to Enhance Checkpoint Blockade Immunotherapy. Angew. Chem. Int. Ed. 2023.
32. Ramasamy, T.; Ruttala, H. B.; Gupta, B.; Poudel, B. K.; Choi, H.-G.; Yong, C. S.; Kim, J. O., Smart chemistry-based nanosized drug delivery systems for systemic applications: a comprehensive review. J. Controlled Release 2017, 258, 226-253.
33. Avgoustakis, K., Pegylated poly (lactide) and poly (lactide-co-glycolide) nanoparticles: preparation, properties and possible applications in drug delivery. Curr. Drug Del. 2004, 1 (4), 321-333.
34. Tadros, M. I.; Al-Mahallawi, A. M., Long-circulating lipoprotein-mimic nanoparticles for smart intravenous delivery of a practically-insoluble antineoplastic drug: development, preliminary safety evaluations and preclinical pharmacokinetic studies. Int. J. Pharm. 2015, 493 (1-2), 439-450.
35. Wegner, E. E.; Adamson, A. W., Photochemistry of complex ions. III. Absolute quantum yields for the photolysis of some aqueous chromium (III) complexes. Chemical actinometry in the long wavelength visible region. J. Am. Chem. Soc. 1966, 88 (3), 394-404.
36. Jana, A.; Devi, K. S. P.; Maiti, T. K.; Singh, N. P., Perylene-3-ylmethanol: fluorescent organic nanoparticles as a single-component photoresponsive nanocarrier with real-time monitoring of anticancer drug release. Journal of the American Chemical Society 2012, 134 (18), 7656-7659.
37. Sasaki, Y.; Amemori, S.; Kouno, H.; Yanai, N.; Kimizuka, N., Near infrared-to-blue photon upconversion by exploiting direct S-T absorption of a molecular sensitizer. Journal of Materials Chemistry C 2017, 5 (21), 5063-5067.
38. Lv, W.; Li, Y.; Li, F.; Lan, X.; Zhang, Y.; Du, L.; Zhao, Q.; Phillips, D. L.; Wang, W., Upconversion-like photolysis of BODIPY-based prodrugs via a one-photon process. J. Am. Chem. Soc. 2019, 141 (44), 17482-17486.

1. A composition, comprising:
a photosensitizer capable of singlet-to-triplet (S-T) activation at a near-infrared (NIR) wavelength; a cleavable moiety to accept triplet-triplet energy transfer from the photosensitizer in a higher energy state to cause cleavage of the cleavable moiety; and a releasable moiety releasable from the composition upon cleavage of the cleavable moiety,
wherein the composition does not comprise an annihilator.

2. (canceled)

3. The composition of claim 1, wherein the single-to-triplet activation is directly from a singlet (S₀) state to a triplet (T₁) state, and the triplet-triplet energy transfer is from the triplet (T₁) state of the photosensitizer to the cleavable moiety.

4. (canceled)

5. The composition of claim 3, wherein the triplet (T₁) state of the photosensitizer is higher than the triplet state of the cleavable moiety.

6. The composition of claim 1, wherein the photosensitizer comprises a transition metal-porphyrin.

7. The composition of claim 6, wherein the transition metal is osmium (Os).

8. The composition of claim 1, wherein the photosensitizer is Os (II) bromophenyl terpyridine complex (Os(bptpy)₂²⁺).

9. The composition of claim 1, wherein the near infrared (NIR) wavelength is between about 650 nm and about 750 nm.

10. The composition of claim 9, wherein the near infrared (NIR) wavelength is about 690 nm.

11. (canceled)

12. (canceled)

13. The composition of claim 1, wherein the composition comprises a prodrug, the prodrug comprising the cleavable moiety which is photocleavable to release the releasable moiety.

14. The composition of claim 13, wherein the cleavable moiety is BODIPY.

15. (canceled)

16. The composition of claim 13, wherein the releasable moiety comprises a drug, and the drug is an anti-inflammatory drug, an anti-cancer drug or an anti-angiogenesis drug.

17. The composition of claim 16, wherein the drug is selected from the group consisting of chlorambucil (CAB), vadimezan, indomethacin, naproxen, ibuprofen, benzyloxycinnamic acid, tetracaine, dopamine, tyramine and homoveratrylamine.

18. (canceled)

19. (canceled)

20. (canceled)

21. The composition of claim 13, wherein the prodrug is represented by the structure: 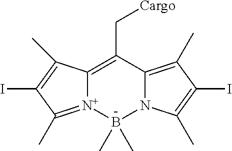
wherein ‘Cargo’ comprises the releasable moiety.

22. The composition of claim 21, wherein Cargo comprises a drug.

23. The composition of claim 13, wherein the prodrug is selected from the group consisting of:

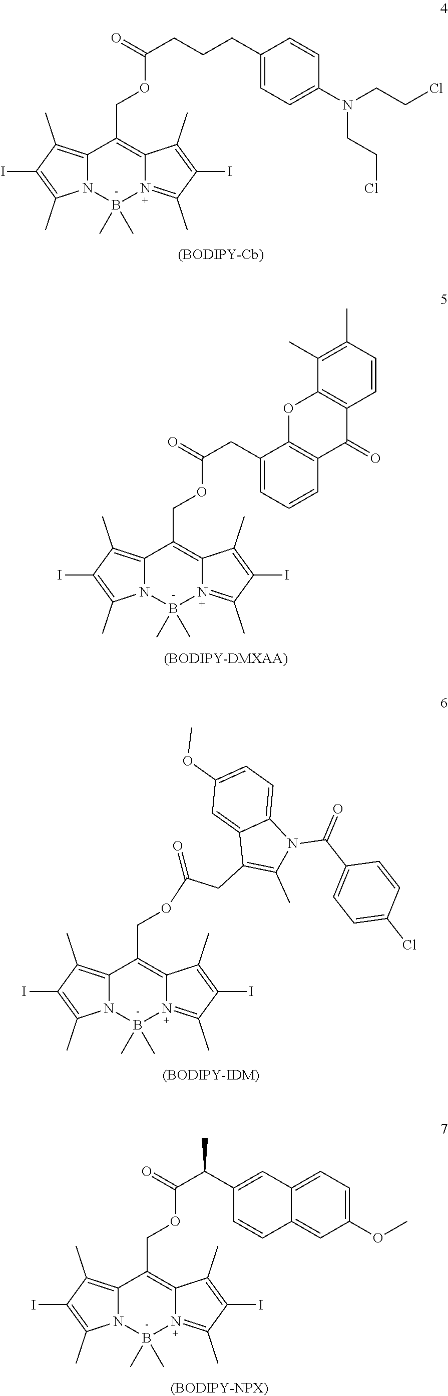

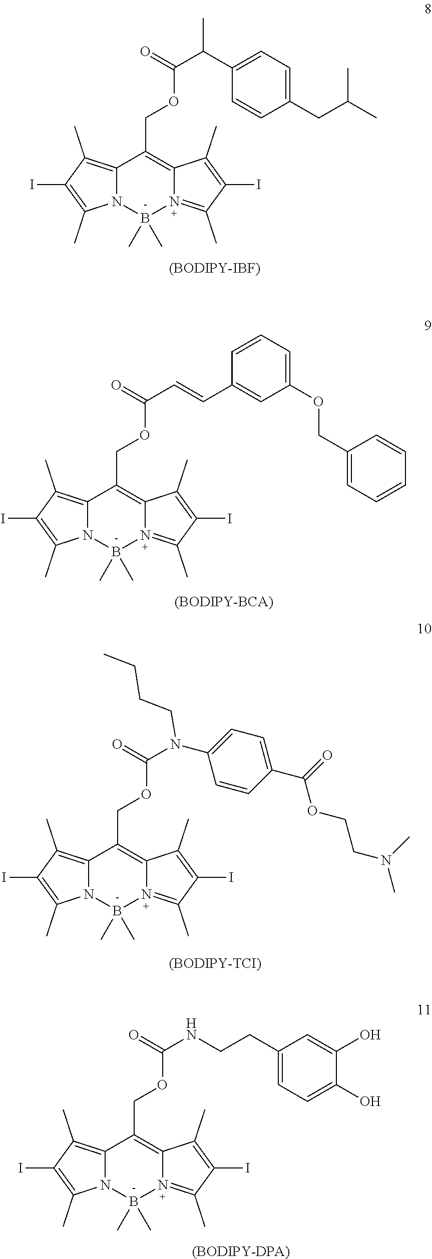

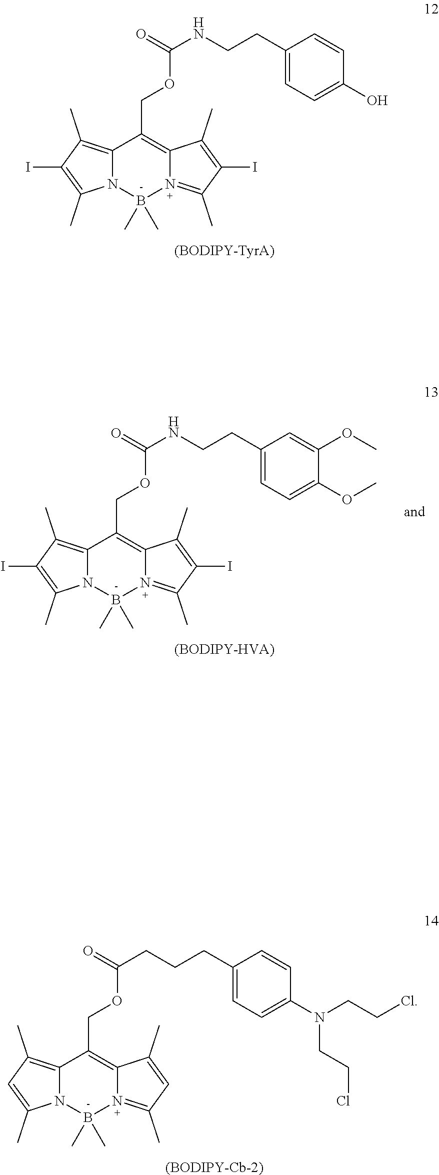

24. The composition of claim 1, wherein at least about 50% of the cleavable moiety is released.

25. The composition of claim 1, wherein at least about 80% of the cleavable moiety is released.

26. (canceled)

27. The composition of claim 1, further comprising a carrier material.

28. The composition of claim 27, wherein the carrier material comprises the photosensitizer, the releasable moiety and the cleavable moiety.

29. The composition of claim 27, wherein the carrier material is formed from a polymer, and optionally present as a particle, a film or a polymeric micelle.

30. (canceled)

31. The composition of claim 29, wherein the particle has an average diameter of less than about 1 mm.

32. (canceled)

33. (canceled)

34. (canceled)

35. A method, comprising:
applying to a subject a composition comprising a photosensitizer capable of singlet-to-triplet (S-T) activation upon exposure to a near-infrared (NIR) wavelength; a cleavable moiety to accept triplet-triplet energy transfer from the photosensitizer in a higher energy state to cause cleavage of the cleavable moiety; and a releasable moiety releasable from the composition upon cleavage of the cleavable moiety; and
applying near-infrared (NIR) light to the subject to cause the cleavage of the cleavable moiety and release of the releasable moiety,
wherein the triplet-triplet energy is not transferred via an annihilator.

36. The method of claim 35, wherein the light is coherent.

37. The method of claim 35, wherein the light is noncoherent.

38. The method of claim 35, wherein the composition comprises a prodrug, the prodrug comprising the cleavable moiety and the releasable moiety.

39. A prodrug comprising a cleavable moiety and a releasable moiety, wherein the cleavable moiety is BODIPY, and wherein the releasable moiety comprises a drug selected from the group consisting of vadimezan, indomethacin, naproxen, ibuprofen, benzyloxycinnamic acid, tetracaine, dopamine, tyramine and homoveratrylamine.

40. The prodrug of claim 39, wherein it has a structure selected from the group consisting of

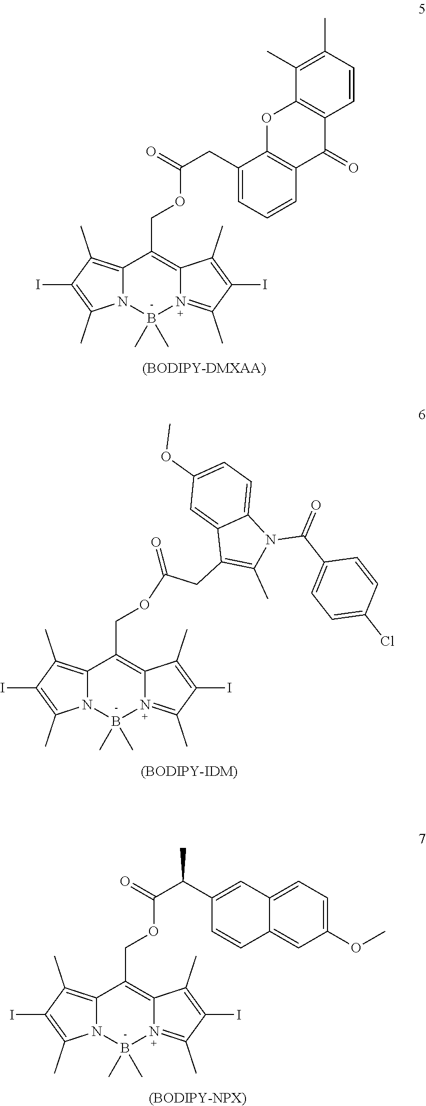

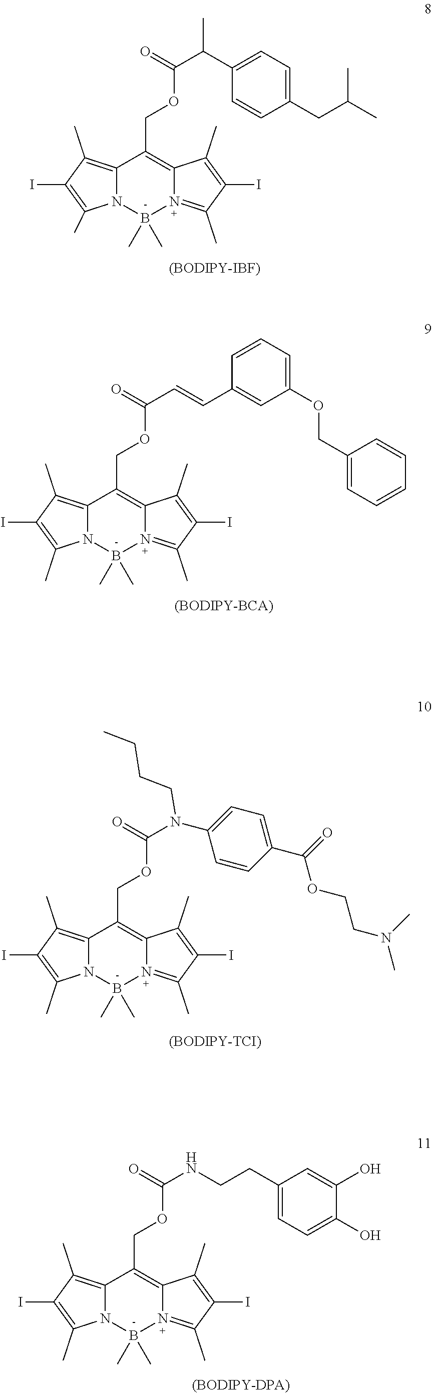

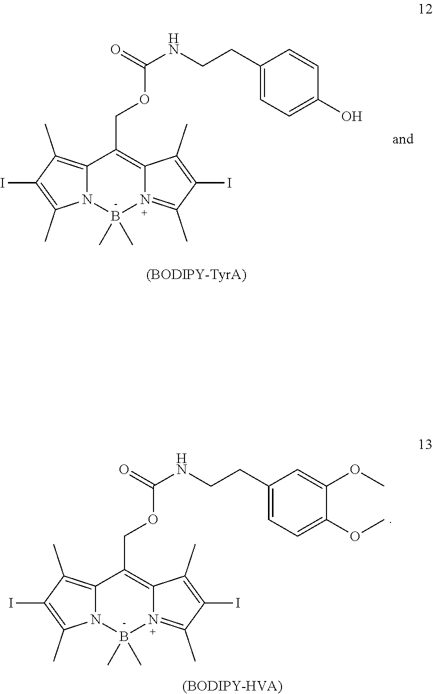

41. The prodrug of claim 40, which is selected from the group consisting of formula 5, 6, 8, and 13.
